(19) United States
(12) Patent Application Publication
(10) Pub. No.: US 2006/0079538 A1
(43) Pub. Date: Apr. 13, 2006

(54) Methods for inhibiting angiogenesis

(76) Inventors: Dennis Hallahan, Nashville, TN (US); Joel S. Hayflick, Seattle, WA (US); Chanchal Sadhu, Bothell, WA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/129,006

(22) Filed: May 12, 2005

Related U.S. Application Data
(60) Provisional application No. 60/570,688, filed on May 13, 2004.

(51) Int. Cl. A61K 31/52

(52) U.S. Cl. 514/263.21

(57) ABSTRACT

The invention relates generally to methods for inhibiting angiogenesis. More particularly, methods for inhibiting angiogenesis comprise selectively inhibiting phosphoinositide 3-kinase delta (PI3Kδ) activity in endothelial cells. The methods may comprise administration of one or more cytotoxic therapies including but not limited to radiation, chemotherapeutic agents, photodynamic therapies, radiofrequency ablation, anti-angiogenic agents, and combinations thereof.

### CROSS-REFERENCE TO RELATED APPLICATION

The benefit under 35 U.S.C. §119(e) of U.S. provisional patent application Ser. No. 60/570,688 filed May 13, 2004, the entire disclosure of which is incorporated herein by reference, is claimed.

### FIELD OF THE INVENTION

The invention relates generally to methods for inhibiting angiogenesis. More particularly, the invention relates to methods for inhibiting angiogenesis comprising selectively inhibiting phosphoinositide 3-kinase delta (PI3Kδ) activity in endothelial cells.

### BACKGROUND OF THE INVENTION

Angiogenesis is the formation of new blood vessels from preexisting ones. Angiogenesis involves multiple steps, including degradation of the originating vessel membrane, endothelial cell migration and proliferation, and formation of new vascular tubules [Ausprunk et al., Microvasc. Res., 14(1):53-65 (1977)]. Typically, angiogenesis is regulated by a balance of endogenous positive and negative angiogenic regulators [Folkman, Nat. Med., 1(1)27-31 (1995); Liekens et al., Biochem. Pharmacol., 61:253-270(2001)].

Angiogenesis is an essential component of normal physiological processes. Angiogenesis is important, for example, in embryo implantation, embryogenesis and development, and wound healing. The vascular endothelium is normally quiescent, however. Thus, angiogenesis is uncommon in healthy adults. More often, angiogenesis is involved in pathological conditions. It is now well recognized that angiogenesis is a component of a large number of otherwise unrelated diseases, conditions, and disorders (hereinafter “indications”), and that such indications can be treated or prevented, or their recurrence can be treated or prevented, by inhibiting angiogenesis. The following discussion provides non-limiting examples of indications involving angiogenesis.

Retinopathy and age-related macular degeneration (AMD), two major causes of vision loss, have been shown to involve angiogenesis. More specifically, these indications typically involve retinal and/or choroidal angiogenesis [Das et al., Prog. Retin. Eye Res., 22(6):721-748 (2003); Grant et al., Drugs Today (Barc.), 38(11):783-791 (2002)]. Anti-angiogenic therapies inhibited retinal and/or choroidal angiogenesis in several animal models, and are therefore considered to have therapeutic value in treating ocular diseases involving angiogenesis [Meneses et al., Gene Ther., 8(8):646-648 (2001); Binétruy-Tournaire et al, EMBO J., 19(7):1525-1533 (2000); see also, Ohno-Matsui et al., Invest. Ophthalmol. Vis. Sci., 44(12):5370-5375 (2003)].

Arthritis is a chronic indication typically involving synovial inflammation, i.e., the inflammation of one or more joints. The onset of synovial inflammation is associated with synovial angiogenesis [Paleolog et al., Angiogenesis, 2(4):295-307 (1998); Clavel et al., Joint Bone Spine, 70(5):321-326 (2003)]. Disrupting synovial angiogenesis is a desirable goal of anti-arthritic therapies, and administration of an anti-angiogenic therapy has reduced the severity of murine collagen-induced arthritis [Sumariwalla et al., Arthritis Res. Ther. 5(1):R32-R39 (2002)].

Psoriasis is a chronic inflammatory skin indication involving angiogenesis that is clinically characterized by the presence of scaly plaques on the skin [Creamer et al., Angiogenesis, 5:231-236 (2002)]. The prominence of psoriatic plaque angiogenesis suggests that psoriasis is angiogenesis-dependent [Barker, Lancet, 338(8761):227-30 (1991)]. Additionally, anti-angiogenic therapy has reduced the severity of psoriasis in humans [Sauder et al., J. Am. Acad. Dermatol., 47(4):535-541 (2002)].

Atherosclerosis involves the deposit of plaques onto arterial walls. Such arterial plaques can rupture, and cause the formation of blood clots capable of causing heart attack and stroke. Plaque angiogenesis has been suggested to promote the progression of atherosclerosis, and anti-angiogenic therapies have inhibited plaque growth in a murine model [Moulton et al., Circ., 99:1726-1732 (1999)].

Endometriosis is an indication in which endometrial cells grow abnormally, i.e., outside of the uterus. The abnormal endometrial cells can cause internal bleeding, inflammation, scarring, and ultimately infertility. Excessive endometrial angiogenesis has been demonstrated in women with endometriosis, and anti-angiogenic therapies have been suggested to have therapeutic potential for treating endometriosis [Healy et-al., Hum. Reprod. Update, 4(5):736-740 (1998)].

Additionally, adipose tissue growth has been shown to be angiogenesis-dependent [Rupnick et al., P.N.A.S., 99:10730-35 (2002)]. Administration of anti-angiogenic therapies in murine obesity models resulted in dose-dependent, reversible weight reduction and adipose tissue loss, and therefore may be applicable for treating, preventing, and/or reversing indications involving excess body fat, such as obesity [Rupnick et al., supra].

Many cancers have been shown to involve angiogenesis. In such cancers, inhibiting angiogenesis may effectively impede the progression of the cancer, or even eradicate the cancer entirely [see, e.g., Bergers et al., Science, 284(5415):808-812 (1999)]. For example, angiogenesis is required for the continuous growth of solid tumors and for tumor metastasis [Folkman, Nat. Med., 1:27-31 (1995)]. Administration of anti-angiogenic therapies inhibited tumor growth in various murine cancer models [Bergers et al., supra; Boehm et al., Nature, 390(6658):404-407 (1997)].

Increased bone marrow angiogenesis occurs in individuals with active multiple myeloma relative to individuals with non-active multiple myeloma [Vacca et al., Neoplasia, 93(9):3064-3073 (1999)]. Furthermore, both circulating and tissue-phase chronic lymphocytic leukemia cells produce and secrete vascular endothelial growth factor (VEGF), a protein known to induce in vivo angiogenesis [Chen et al., Neoplasia, 96(9):3181-3187 (2000)].

Elevated levels of basic fibroblast growth factor (bFGF), another protein known to induce in vivo angiogenesis, have been detected in individuals having non-Hodgkin's lymphoma [Salven et al., Blood, 94(10):3334-3339 (1999)]. Thus, anti-angiogenic therapies have been proposed for treatment of hematological cancers including but not limited to leukemia, multiple myeloma, and lymphomas [Moehler et al., Ann. Hematol. 80(12):695-705 (2001)].

Additionally, angiogenesis appears to be important both in the pathogenesis of acute myelogenous leukemia (AML) and for the susceptibility of AML blasts to chemotherapy [Glenjen et al., Int J cancer.101(1):86-94 (2002)]. Thus, inhibiting angiogenesis could constitute a strategy for treating AML [Hussong et al., Blood. 95(1):309-13 (2000)].

Cancers generally include solid tumors, hematological cancers (including but not limited to multiple myeloma and leukemias), and lymphomas. Cancers are caused by cancerous cells, i.e., cells that multiply uncontrollably. Cancer is typically treated with one or more therapies including but not limited to surgery, radiation therapy, chemotherapy, and immunotherapy. Surgery involves the bulk removal of diseased tissue. While surgery can be effectively used to remove certain tumors, it cannot be used to treat tumors located in areas that are inaccessible to surgeons. Additionally, surgery cannot be successfully used to treat non-localized cancerous indications including but not limited to leukemia and multiple myeloma.

Radiation therapy involves using high-energy radiation from x-rays, gamma rays, neutrons, and other sources (“radiation”) to kill cancerous cells and shrink tumors. Radiation therapy is well known in the art [Hellman, Cancer: Principles and Practice of Oncology, 248-75, 4^{th} ed., vol. 1 (1993)]. Radiation therapy may be administered from outside the body (“external-beam radiation therapy”). Alternatively, radiation therapy can be administered by placing radioactive materials capable of producing radiation in or near the tumor or in an area near the cancerous cells. Systemic radiation therapy employs radioactive substances including but not limited to radiolabeled monoclonal antibodies that can circulate throughout the body or localize to specific regions or organs of the body. Brachytherapy involves placing a radioactive “seed” in proximity to a tumor. Radiation therapy is non-specific and often causes damage to any exposed tissues. Additionally, radiation therapy frequently causes individuals to experience side effects (such as nausea, fatigue, low leukocyte counts, etc.) that can significantly affect their quality of life and influence their continued compliance with radiation treatment protocols. Radiation therapy is typically employed as a potentially curative therapy for individuals who have a clinically localized cancer and are expected to live at least about five years without treatment.

The response of tumor microvasculature to radiation is dependent upon the dose and time interval after treatment [Johnson et al., Intl. J. Rad. Onc. Biol. Phys., 1:659-670 (1976); Hilmas et al., Rad. Res., 61:128-143 (1975); Kallman et al., Canc. Res., 32:483-490 (1972); Yamaura et al., Int. J. Rad. Biol., 30:179-187 (1976); Ting et al., Int. J. Rad. Biol., 60: 335-339, 1991; Song et al., Canc. Res., 34:2344-2350 (1974)]. For example, tumor blood flow decreases when tumors are treated with doses in the range of 20 to 45 Gy [Song et al., supra], and tumor blood volume increases if doses below 500 rads are administered [Johnson et al., supra; Kallman et al., supra]. Additionally, blood flow studies of irradiated mouse sarcoma show that blood flow increases within 3 to 7 days of treatment [Kallman et al., supra]. Tumor blood vessels show less response to radiation doses in the range of 2-3 Gy, which are used during conventional radiation therapy [Geng et al., Canc. Res., 61(6):2413-19 (2001); Edwards et al., Canc. Res., 62:4671-7 (2002); Schueneman et al., Canc. Res., 63:4009-16 (2003)]. Radiation doses in the range of 6 Gy are required to achieve tumor vascular destruction [Garcia-Barros et al., Science, 300(5622):1155-59 (2003); Geng et al., supra; Schueneman et al., supra].

Chemotherapy involves administering chemotherapeutic agents, which act by disrupting cell replication or cell metabolism (e.g., by disrupting DNA metabolism, DNA synthesis, DNA transcription, or microtubule spindle function, or by perturbing chromosomal structural integrity by way of introducing DNA lesions). Chemotherapeutics are frequently non-specific in that they can affect normal healthy cells as well as tumor cells. The maintenance of DNA integrity is essential to cell viability in normal cells. Therefore, chemotherapeutics typically have very low therapeutic indices, i.e., the window between the effective dose and the excessively toxic dose can be extremely narrow because the drugs cause a high rate of damage to normal cells as well as tumor cells. Additionally, chemotherapy-induced side effects significantly affect the quality of life of an individual in need of treatment, and therefore frequently influence the individual's continued compliance with chemotherapy treatment protocols. Chemotherapy is used most often to treat breast, lung, and testicular cancer.

Cellular immune deficiency and tumor-associated immune suppression are linked with various cancers [Hadden, Int. Immunopharmacol. 3(8):1061-1071 (2003)]. Consequently, immunotherapeutics, i.e., compositions comprising cytokines, growth factors, antigens, and/or antibodies have been proposed for treating cancers [Hadden, supra; Cebon et al., Cancer Immun., 16(3):7-25 (2003)].

Other cancer therapies are also known. For example, photodynamic therapy (PDT) involves the administration of a photosensitizing compound or drug, typically orally, intravenously, or topically, that can be activated by an external light source to destroy a target tissue. The photosensitizing drug itself is harmless and rapidly leaves normal cells, but it remains in rapidly proliferating cells including but not limited to cancer cells for a longer time. Typically, a laser is then aimed at a tumor (or other cell mass), thereby activating the photosensitizing drug and killing the cells that have absorbed it. Photodynamic therapy is typically used to treat very small tumors in individuals. It is also known for use in treatment of psoriasis.

Radiofrequency ablation is a minimally invasive treatment involving the insertion of a catheter device into a tumor. The catheter device is guided by imaging techniques and includes an electrode capable of transmitting radiofrequency energy disposed along the catheter device tip. Tissues in proximity to the catheter device tip are exposed to the radiofrequency energy and localized cytotoxicity results from the heating effect caused by the transmitted radiofrequency energy [Johnson et al., J. Endourol. 17(8):557-62 (2003); Chang, BioMed. Eng. Online, 2:12 (2003)]. Radiation frequency ablation is advantageous in that the catheter device can be inserted in surgically inaccessible tumors. Radiation frequency ablation is most frequently used to treat small tumors including cancers of the liver.

Anti-angiogenic therapies for cancer have been demonstrated in combination with radiation therapy. The response of tumor blood vessels to radiation therapy is enhanced by administration of inhibitors of receptor tyrosine kinases (RTK) [Geng et al., supra; Schueneman et al., supra; Gorski et al., Canc. Res., 59:3374-3378 (1999)]. RTK inhibitors administered prior to irradiation attenuated Akt-phosphorylation in vascular endothelium and improved tumor growth delay in response to radiation [Geng et al., supra; Schueneman et al., supra; Gorski et al., supra].

The anti-angiogenic methods of the invention relate to selectively inhibiting phosphoinositide 3-kinase delta (PI3Kδ) activity in endothelial cells. The following discussion relates to phosphoinositide 3-kinases (PI3Ks).

Phosphorylation of Akt has been widely used as an indirect measure of Class I PI3K activity in multiple cell types, including human umbilical vein endothelial cells (HUVECs) [Shiojima et al., Circ. Res., 90:1243-1250 (2002); Kandel et al., Exp. Cell Res., 253:210-229 (1999); Cantley et al., Science, 296:1655-1657 (2002)]. PI3K activity is required for growth factor mediated survival of various cell types [Fantl et al., Ann. Rev. Biochem., 62:453-81 (1993); Datta et al., Genes & Dev., 13(22):2905-27 (1999)].

PI3Ks catalyze the addition of a phosphate group to the inositol ring of phosphoinositides [Wymann et al., Biochim. Biophys. Acta, 1436:127-150 (1998)]. One target of these phosphorylated products is the serine/threonine protein kinase B (PKB or Akt). Akt subsequently phosphorylates several downstream targets, including the Bcl-2 family member Bad and caspase-9, thereby inhibiting their pro-apoptotic functions [Datta et al., Cell 91: 231-41, (1997); Cardone et al., Science 282: 1318-21, (1998)]. Akt has also been shown to phosphorylate the forkhead transcription factor FKHR [Tang et al., J. Biol. Chem., 274:16741-6 (1999)]. In addition, many other members of the apoptotic machinery as well as transcription factors contain the Akt consensus phosphorylation site [Datta et al., supra].

Structurally, PI3Ks exist as heterodimeric complexes, consisting of a p110 catalytic subunit and a p55, p85, or p101 regulatory subunit. There are four different p110 catalytic subunits, which are classified as p110α, p110β, p110γ, and p110δ [Wymann et al., Biochim. Biophys. Acta, 1436:127-150 (1998); Vanhaesebroeck et al., Trends Biochem. Sci., 22:267-272 (1997)].

The nonselective phosphoinositide 3-kinase (PI3K) inhibitors, LY294002 and wortmannin, have been shown to enhance destruction of tumor vasculature in irradiated endothelial cells [Edwards et al., Cancer Res., 62: 4671-7 (2002)]. LY294002 and wortmannin do not distinguish among the four members of class I PI3Ks. For example, the IC₅₀ values of wortmannin against each of the various class I PI3Ks are in the range of 1-10 nM. Similarly, the IC₅₀ values for LY294002 against each of these PI3Ks is about 1 μM [Fruman et al., Ann. Rev. Biochem., 67:481-507 (1998)]. These inhibitors are not only nonselective with respect to class I PI3Ks, but are also potent inhibitors of DNA dependent protein kinase, FRAP-mTOR, smooth muscle myosin light chain kinase, and casein kinase 2 [Hartley et al., Cell 82:849 (1995); Davies et al., Biochem. J. 351:95 (2000); Brunn et al., EMBO J. 15:5256 (1996)].

Because p110α, p110β, p110γ, and p110δ are expressed differentially by a wide variety of cell types, the administration of nonselective PI3K inhibitors such as LY294002 and wortmannin almost certainly will also affect cell types that may not be targeted for treatment. Therefore, the effective therapeutic dose of such nonselective inhibitors would be expected to clinically unusable because otherwise non-targeted cell types will likely be affected, especially when such nonselective inhibitors are combined with cytotoxic therapies including but not limited to chemotherapy, radiation therapy, photodynamic therapies, radiofrequency ablation, and/or anti-angiogenic therapies.

Therefore, important and significant goals are to develop and make available safer and more effective methods of treating and preventing indications involving angiogenesis, and to provide cancer and other therapies that facilitate clinical management and continued compliance of the individual being treated with treatment protocols.

### SUMMARY OF THE INVENTION

The invention provides methods for inhibiting angiogenesis comprising selectively inhibiting phosphoinositide 3-kinase delta (PI3Kδ) activity in endothelial cells to inhibit angiogenesis. In one aspect of this embodiment, the methods comprise administering an amount of a phosphoinositide 3-kinase delta (PI3Kδ) selective inhibitor effective to inhibit angiogenesis.

In another embodiment, the invention provides methods for inhibiting endothelial cell migration comprising selectively inhibiting phosphoinositide 3-kinase delta (PI3Kδ) activity in endothelial cells to inhibit endothelial cell migration. In one aspect of this embodiment, the methods comprise administering an amount of a phosphoinositide 3-kinase delta (PI3Kδ) selective inhibitor effective to inhibit endothelial cell migration.

In an additional embodiment, the invention provides methods for inhibiting tumor growth comprising selectively inhibiting phosphoinositide 3-kinase delta (PI3Kδ) activity in endothelial cells to inhibit tumor growth. In one aspect of this embodiment, the methods comprise administering an amount of a PI3Kδ selective inhibitor effective to inhibit tumor growth.

In a further embodiment, the invention provides methods for reducing tumor vasculature formation or repair comprising selectively inhibiting phosphoinositide 3-kinase delta (PI3Kδ) activity in endothelial cells to reduce tumor vasculature formation or repair. In one aspect of this embodiment, the methods comprise administering an amount of a PI3Kδ selective inhibitor effective to reduce tumor vasculature formation or repair.

In another embodiment, the invention provides methods for inhibiting endothelial tubule formation comprising selectively inhibiting phosphoinositide 3-kinase delta (PI3Kδ) activity in endothelial cells to inhibit endothelial tubule formation. In one aspect of this embodiment, the methods comprise administering an amount of a PI3Kδ selective inhibitor effective to inhibit endothelial tubule formation.

In an additional embodiment, the invention provides methods for reducing tumor mass comprising selectively inhibiting phosphoinositide 3-kinase delta (PI3Kδ) activity in endothelial cells to reduce tumor mass. In one aspect of this embodiment, the methods comprise administering an amount of a PI3Kδ selective inhibitor effective to reduce tumor mass.

In a further embodiment, the invention provides methods for treating or preventing an indication involving angiogenesis comprising selectively inhibiting phosphoinositide 3-kinase delta (PI3Kδ) activity in endothelial cells to inhibit angiogenesis in an individual in need thereof. In one aspect of this embodiment, the methods comprise administering an amount of a PI3Kδ selective inhibitor effective to inhibit angiogenesis in an individual in need thereof.

In an additional embodiment, the invention provides methods for enhancing apoptosis in endothelial cells comprising selectively inhibiting phosphoinositide 3-kinase delta (PI3Kδ) activity in endothelial cells. One aspect according to this embodiment provides methods for enhancing apoptosis in endothelial cells comprising administering an amount of a PI3Kδ selective inhibitor effective to enhance apoptosis in endothelial cells. Another aspect provides methods for enhancing apoptosis in endothelial cells comprising administering a therapeutically effective amount of a combination comprising a PI3Kδ selective inhibitor and radiation to enhance apoptosis in endothelial cells. In another aspect, the invention provides methods for enhancing apoptosis in endothelial cells comprising administering a therapeutically effective amount of a combination comprising a PI3Kδ selective inhibitor and a chemotherapeutic agent to enhance apoptosis in endothelial cells. A further aspect of the invention provides methods for enhancing apoptosis in endothelial cells comprising administering a therapeutically effective amount of a PI3Kδ selective inhibitor alone or a combination comprising a PI3Kδ selective inhibitor, a photosensitizing compound, and light (typically, long wavelength UV light) to enhance apoptosis in endothelial cells. In a still further aspect, the invention provides methods for enhancing apoptosis in endothelial cells comprising administering a therapeutically effective amount of a PI3Kδ selective inhibitor alone or a combination comprising a PI3Kδ selective inhibitor and radiofrequency energy (pursuant to a radiofrequency ablation therapy protocol) to enhance apoptosis in endothelial cells. In another aspect, the invention provides methods for enhancing apoptosis in endothelial cells comprising administering a therapeutically effective amount of a combination comprising a PI3Kδ selective inhibitor and an anti-angiogenic agent, optionally in combination with one or more of the above-mentioned types of agents, to enhance apoptosis in endothelial cells.

In yet another embodiment, the invention provides methods for increasing the therapeutic indices of cytotoxic cancer therapies. In one aspect according to this embodiment, the invention provides methods for increasing the therapeutic index of radiation comprising administering a combination comprising radiation and an amount of a PI3Kδ selective inhibitor effective to increase the therapeutic index of radiation. In another aspect, the invention provides methods for increasing the therapeutic index of a chemotherapeutic agent comprising administering a combination comprising a chemotherapeutic agent and an amount of a PI3Kδ selective inhibitor effective to increase the therapeutic index of the chemotherapeutic agent. In a further aspect, the invention provides methods for increasing the therapeutic index of photodynamic therapy comprising administering a combination comprising a photosensitizing compound, light, and an amount of a PI3Kδ selective inhibitor effective to increase the therapeutic index of the photodynamic therapy. In yet a further aspect, the invention provides methods for increasing the therapeutic index of an anti-angiogenic agent comprising administering a combination comprising an anti-angiogenic agent and an amount of a PI3Kδ selective inhibitor effective to increase the therapeutic index of the anti-angiogenic agent.

In a further embodiment, the invention provides methods for reducing highly vascularized tissues comprising selectively inhibiting phosphoinositide 3-kinase delta (PI3Kδ) activity in endothelial cells to reduce vascular growth or vascular repair of a highly vascularized tissue. In one aspect of this embodiment, the methods comprise administering an amount of a PI3Kδ selective inhibitor effective to reduce vascular growth or vascular repair of a highly vascularized tissue. In another aspect of this embodiment, the highly vascularized tissue is adipose tissue. In yet another aspect, the highly vascularized tissue is retinal tissue.

### DETAILED DESCRIPTION

Angiogenesis involves multiple steps, including degradation of the originating vessel membrane, endothelial cell migration and proliferation, and formation of new vascular tubules [Ausprunk et al., Microvasc. Res., 14(1):53-65 (1977)]. Suppressing any one of these steps inhibits angiogenesis. Additionally, endothelial progenitor cells are present in bone marrow and can be activated and recruited to contribute to angiogenesis [Quirici et al., Br. J. Haematol. 115(1):186-194 (2001); Reyes et al., J. Clin. Invest., 109(3):313-315 (2002); Annabi et al., J. Cell. Biochem. 91(6):1146-1158 (2004)]. Suppressing the activation and recruitment of such progenitor cells also inhibits angiogenesis.

The invention provides methods for inhibiting angiogenesis comprising selectively inhibiting phosphoinositide 3-kinase delta (PI3Kδ) activity in endothelial cells to inhibit angiogenesis. Thus, the methods of the invention include inhibiting angiogenesis by inhibiting an upstream target in the pathway that selectively inhibits PI3Kδ. In one aspect of this embodiment, the methods comprise administering an amount of a phosphoinositide 3-kinase delta (PI3Kδ) selective inhibitor effective to inhibit angiogenesis.

As used herein, the term “selectively inhibiting phosphoinositide 3-kinase delta (PI3Kδ) activity” generally refers to inhibiting the activity of the PI3Kδ isozyme more effectively than other isozymes of the PI3K family. Similarly, the term “PI3Kδ selective inhibitor” generally refers to a compound that inhibits the activity of the PI3Kδ isozyme more effectively than other isozymes of the PI3K family. A PI3Kδ selective inhibitor compound is therefore more selective for PI3Kδ than conventional PI3K inhibitors such as wortmannin and LY294002, which are “nonselective PI3K inhibitors.”

As used herein, the term “amount effective” means a dosage sufficient to produce a desired or stated effect.

In another embodiment, the invention provides methods for inhibiting endothelial cell migration comprising selectively inhibiting phosphoinositide 3-kinase delta (PI3Kδ) activity in endothelial cells to inhibit endothelial cell migration. In one aspect of this embodiment, the methods comprise administering an amount of a phosphoinositide 3-kinase delta (PI3Kδ) selective inhibitor effective to inhibit endothelial cell migration.

In an additional embodiment, the invention provides methods for inhibiting tumor growth comprising selectively inhibiting phosphoinositide 3-kinase delta (PI3Kδ) activity in endothelial cells to inhibit tumor growth. In one aspect of this embodiment, the methods comprise administering an amount of a PI3Kδ selective inhibitor effective to inhibit tumor growth.

In a further embodiment, the invention provides methods for reducing tumor vasculature formation or repair comprising selectively inhibiting phosphoinositide 3-kinase delta (PI3Kδ) activity in endothelial cells to reduce tumor vasculature formation or repair. In one aspect of this embodiment, the methods comprise administering an amount of a PI3Kδ selective inhibitor effective to reduce tumor vasculature formation or repair.

In another embodiment, the invention provides methods for inhibiting endothelial tubule formation comprising selectively inhibiting phosphoinositide 3-kinase delta (PI3Kδ) activity in endothelial cells to inhibit endothelial tubule formation. In one aspect of this embodiment, the methods comprise administering an amount of a PI3Kδ selective inhibitor effective to inhibit endothelial tubule formation.

In an additional embodiment, the invention provides methods for reducing tumor mass comprising selectively inhibiting phosphoinositide 3-kinase delta (PI3Kδ) activity in endothelial cells to reduce tumor mass. In one aspect of this embodiment, the methods comprise administering an amount of a PI3Kδ selective inhibitor effective to reduce tumor mass.

In a further embodiment, the invention provides methods for treating or preventing an indication involving angiogenesis comprising selectively inhibiting phosphoinositide 3-kinase delta (PI3Kδ) activity in endothelial cells to inhibit angiogenesis in an individual in need thereof. In one aspect of this embodiment, the methods comprise administering an amount of a PI3Kδ selective inhibitor effective to inhibit angiogenesis in an individual in need thereof.

In an additional embodiment, the invention provides methods for enhancing apoptosis of endothelial cells comprising selectively inhibiting phosphoinositide 3-kinase delta (PI3Kδ) activity in endothelial cells. One aspect according to this embodiment provides methods for enhancing apoptosis in endothelial cells comprising administering an amount of a PI3Kδ selective inhibitor effective to enhance apoptosis in endothelial cells. Another aspect according to this embodiment provides methods for enhancing apoptosis in endothelial cells comprising administering a therapeutically effective amount of a combination comprising a PI3Kδ selective inhibitor and radiation to enhance apoptosis in endothelial cells.

As used herein, the term “therapeutically effective amount” refers to a dosage sufficient to produce a desired or stated effect.

As used herein, the term “radiation” refers to high energy radiation capable of inducing DNA damage within cells, including but not limited to gamma-rays, X-rays, high energy electrons, and protons.

In another aspect, the invention provides methods for enhancing apoptosis in endothelial cells comprising administering a therapeutically effective amount of a combination comprising a PI3Kδ selective inhibitor and a chemotherapeutic agent to enhance apoptosis in endothelial cells.

As used herein, the term “chemotherapeutic agent” refers to a drug that destroys cancer cells by stopping them from growing or multiplying.

A further aspect of the invention provides methods for enhancing apoptosis in endothelial cells comprising administering a therapeutically effective amount of a combination comprising a PI3Kδ selective inhibitor, a photosensitizing compound, and light (typically, long wavelength UV light) to enhance apoptosis in endothelial cells.

As used herein, the term “photosensitizing compound” refers to a compound administered in an unactive, harmless form that can be activated by an external light source to destroy a target tissue.

In a still further aspect, the invention provides methods for enhancing apoptosis in endothelial cells comprising administering a therapeutically effective amount of a combination comprising a PI3Kδ selective inhibitor and radiofrequency energy (pursuant to a radiofrequency ablation therapy protocol) to enhance apoptosis in endothelial cells.

As used herein, “radiofrequency energy” refers to non-ionizing electromagnetic radiation capable of causing an increase in temperature (similar to microwave energy).

In another aspect, the invention provides methods for enhancing apoptosis in endothelial cells comprising administering a therapeutically effective amount of a combination comprising a PI3Kδ selective inhibitor and an anti-angiogenic agent to enhance apoptosis in endothelial cells.

In yet another embodiment, the invention provides methods for increasing the therapeutic indices of cytotoxic cancer therapies.

As used herein, “therapeutic index” is a dose ratio between toxic and therapeutic effects that is expressed as the ratio of LD50 to ED50.

As used herein, the term “cytotoxic therapy” as used herein refers to therapies that induce cellular damage including but not limited to radiation, chemotherapy, photodynamic therapy, radiofrequency ablation, anti-angiogenic therapy, and combinations thereof. A cytotoxic therapeutic may induce DNA damage when applied to a cell, as described below.

As used herein, the term “DNA damaging agents” include compounds and treatment methods that induce DNA damage when applied to a cell. Such agents include but are not limited to radiation, DNA-damaging chemotherapeutic agents, and photosensitizing agents which have been activated (pursuant to a PDT therapy protocol).

In one aspect according to this embodiment, the invention provides methods for increasing the therapeutic index of radiation comprising administering a combination comprising radiation and an amount of a PI3Kδ selective inhibitor effective to increase the therapeutic index of radiation.

In another aspect according to this embodiment, the invention provides methods for increasing the therapeutic index of a chemotherapeutic agent comprising administering a combination comprising a chemotherapeutic agent and an amount of a PI3Kδ selective inhibitor effective to increase the therapeutic index of the chemotherapeutic agent.

In a further aspect according to this embodiment, the invention provides methods for increasing the therapeutic index of photodynamic therapy comprising administering a combination comprising a photosensitizing compound, light, and an amount of a PI3Kδ selective inhibitor effective to increase the therapeutic index of the photodynamic therapy.

In yet a further aspect according to this embodiment, the invention provides methods for increasing the therapeutic index of an anti-angiogenic agent comprising administering a combination comprising an anti-angiogenic agent and an amount of a PI3Kδ selective inhibitor effective to increase the therapeutic index of the anti-angiogenic agent.

Throughout the specification, methods that include administration of a PI3Kδ selective inhibitor and administration of one or more cytotoxic therapies including but not limited to radiation, a chemotherapeutic agent, photodynamic therapy, radiofrequency ablation, an anti-angiogenic agent, and combinations thereof, are generally referred to as “combination methods in accordance with the invention.”

The cytotoxic therapies used for cancer treatment can be administered in the combination methods according to the invention at a low dose, that is, at a dose lower than conventionally used in clinical situations where the cytotoxic therapeutic is administered alone, because the PI3Kδ selective nature of the inhibitors of the invention increases the therapeutic index (i.e., the specificity) of the inventive combination therapies. Lowering the dose of the cytotoxic therapeutic administered to an individual decreases the incidence of adverse effects associated with higher dosages, and can thereby improve the quality of life of an individual undergoing treatment. Further benefits include improved compliance with the treatment protocol of the individual being treated, and a reduction in the number of hospitalizations needed for the treatment of adverse effects. Additionally, the specificity of the methods of the invention are advantageous in that they permit treatment at higher doses of the PI3Kδ selective inhibitor(s) than nonselective inhibitors such as LY294002 and wortmannin, further maximizing the therapeutic efficacy of the inventive methods.

In a further embodiment, the invention provides methods for reducing highly vascularized tissues comprising selectively inhibiting phosphoinositide 3-kinase delta (PI3Kδ) activity in endothelial cells to reduce vascular growth or vascular repair of a highly vascularized tissue. In one aspect of this embodiment, the methods comprise administering an amount of a PI3Kδ selective inhibitor effective to reduce vascular growth or vascular repair of a highly vascularized tissue. In another aspect of this embodiment, the highly vascularized tissue is adipose tissue. In yet another aspect, the highly vascularized tissue is retinal tissue.

As previously described, the term “PI3Kδ selective inhibitor” generally refers to a compound that inhibits the activity of the PI3Kδ isozyme more effectively than other isozymes of the PI3K family. The relative efficacies of compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC50.” IC50 determinations can be accomplished using conventional techniques known in the art. In general, an IC50 can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC50 value. Analogously, other inhibitory concentrations can be defined through appropriate determinations of activity. For example, in some settings it can be desirable to establish a 90% inhibitory concentration, i.e., IC90, etc.

Accordingly, a PI3Kδ selective inhibitor alternatively can be understood to refer to a compound that exhibits a 50% inhibitory concentration (IC50) with respect to PI3Kδ that is at least 10-fold, in another aspect at least 20-fold, and in another aspect at least 30-fold, lower than the IC50 value with respect to any or all of the other class I PI3K family members. In an alternative embodiment of the invention, the term PI3Kδ selective inhibitor can be understood to refer to a compound that exhibits an IC50 with respect to PI3Kδ that is at least 50-fold, in another aspect at least 100-fold, in an additional aspect at least 200-fold, and in yet another aspect at least 500-fold, lower than the IC50 with respect to any or all of the other PI3K class I family members. A PI3Kδ selective inhibitor is typically administered in an amount such that it selectively inhibits PI3Kδ activity, as described above.

Any selective inhibitor of PI3Kδ activity, including but not limited to small molecule inhibitors, peptide inhibitors, non-peptide inhibitors, naturally occurring inhibitors, and synthetic inhibitors, may be used in the methods. Suitable PI3Kδ selective inhibitors have been described in U.S. Patent Publication 2002/161014 to Sadhu et al., the entire disclosure of which is hereby incorporated herein by reference. Compounds that compete with a PI3Kδ selective inhibitor compound described herein for binding to PI3Kδ and selectively inhibit PI3Kδ are also contemplated for use in the methods of the invention. Methods of identifying compounds which competitively bind with PI3Kδ, with respect to the PI3Kδ selective inhibitor compounds specifically provided herein, are well known in the art [see, e.g., Coligan et al., Current Protocols in Protein Science, A.5A.15-20, vol. 3 (2002)]. In view of the above disclosures, therefore, PI3Kδ selective inhibitor embraces the specific PI3Kδ selective inhibitor compounds disclosed herein, compounds having similar inhibitory profiles, and compounds that compete with the such PI3Kδ selective inhibitor compounds for binding to PI3Kδ, and in each case, conjugates and derivatives thereof.

The methods of the invention may be applied to cell populations in vivo or ex vivo. “In vivo” means within a living individual, as within an animal or human. In this context, the methods of the invention may be used therapeutically in an individual, as described infra. The methods may also be used prophylactically.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. For example, the invention may be used ex vivo to determine the optimal schedule and/or dosing of administration of a PI3Kδ selective inhibitor for a given indication, cell type, individual, and other parameters. Information gleaned from such use may be used for experimental purposes or in the clinic to set protocols for in vivo treatment. Other ex vivo uses for which the invention may be suited are described below or will become apparent to those skilled in the art.

The methods in accordance with the invention can be used to treat any indication involving angiogenesis, as the methods of the invention inhibit the formation of the vasculature formed pursuant to angiogenesis. In one aspect, the methods inhibit the formation of the vasculature that supplies cancerous cells with blood and nutrients. Treatment may be of any cancerous indication, including cancers that present as a solid tumor mass, and other cancers that typically do not present as a tumor mass, but are distributed in the vascular or lymphoreticular systems.

Cancers that present as solid tumors that involve angiogenesis and are treatable by the methods of the invention include but are not limited to carcinomas and sarcomas. Carcinomas derive from epithelial cells which infiltrate (i.e., invade) surrounding tissues and give rise to metastases. Adenocarcinomas are carcinomas derived from glandular tissue, or from tissues that form recognizable glandular structures. Sarcomas are tumors whose cells are embedded in a fibrillar or homogeneous substance, like embryonic connective tissue. Cancers that typically do not present as solid tumors and are treatable by the methods of the invention include but are not limited to lymphomas and hematological cancers including but not limited to myelomas and leukemias.

The methods of the invention also provide for the treatment of cancers including but not limited to myxoid and round cell carcinomas, human soft tissue sarcomas including Ewing's sarcoma, cancer metastases including lymphatic metastases, squamous cell carcinomas (particularly of the head and neck), esophageal squamous cell carcinomas, oral carcinomas, blood cell malignancies (including multiple myelomas), leukemias (including acute lymphocytic leukemias), acute nonlymphocytic leukemias, chronic lymphocytic leukemias, chronic myelocytic leukemias, and hairy cell leukemias, effusion lymphomas (i.e., body cavity-based lymphomas), thymic lymphoma lung cancers (including small cell carcinomas of the lungs), cutaneous T cell lymphomas, Hodgkin's lymphomas, non-Hodgkin's lymphomas, cancers of the adrenal cortex, ACTH-producing tumors, non-small cell lung cancers, breast cancers (including small cell carcinomas and ductal carcinomas), gastro-intestinal cancers (including stomach cancers, colon cancers, colorectal cancers, and polyps associated with colorectal neoplasias), pancreatic cancers, liver cancers, urological cancers (including but not limited to bladder cancers such as primary superficial bladder tumors, invasive transitional cell carcinomas of the bladder, and muscle-invasive bladder cancers), malignancies of the female reproductive tract (including ovarian carcinomas, primary peritoneal epithelial neoplasms, cervical carcinomas, uterine endometrial cancers, vaginal cancers, cancers of the vulva, uterine cancers and solid tumors in the ovarian follicle), malignancies of the male reproductive tract (including testicular cancers, penile cancers and prostate cancers), kidney cancers (including renal cell carcinomas), brain cancers (including intrinsic brain tumors, neuroblastomas, astrocytic brain tumors, gliomas, and metastatic tumor cell invasions in the central nervous system), bone cancers (including osteomas and osteosarcomas), skin cancers (including malignant melanomas, tumor progressions of human skin keratinocytes, basal cell carcinomas, and squamous cell cancers), thyroid cancers, retinoblastomas, neuroblastomas, peritoneal effusions, malignant pleural effusions, mesotheliomas, Wilms's tumors, gall bladder cancers, trophoblastic neoplasms, hemangiopericytomas, and Kaposi's sarcomas.

The methods of the invention are also contemplated in treatment of non-cancerous indications involving angiogenesis. Such indications include but are not limited to retinopathy, age-related macular degeneration (AMD), arthritis, psoriasis, atherosclerosis, and endometriosis.

Animal models of some of the foregoing cancerous and non-cancerous indications treatable by the invention include for example: viable cancer cells from the HL60 cell line (human non-small cell lung cancer) injected into athymic nude mice, Panc-01 human tumor cells (human pancreatic cancer) injected into athymic nude mice, A375 human tumor cells (human melanoma) injected into athymic nude mice, SKMES lung cancer cells (human lung cancer) injected into athymic nude mice, SKOV-3.ip. ovarian carcinoma cells (human ovarian cancer) injected into athymic nude mice, MDA-MB-361 breast cancer cells (human breast cancer) injected into athymic nude mice, 137-62 cells (breast cancer) injected into rats, metalloproteinase-2 deficient (MMP-2(−/−) mice (ocular disease involving angiogenesis), rabbit corneal stroma injected with slow releasing implants containing VEGF (ocular disease involving angiogenesis), bovine collagen injected into mice (arthritis), and apolipoprotein E-deficient (apoE −/−) mice (atherosclerosis).

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The methods in accordance with the invention may include administering a PI3Kδ selective inhibitor with one or more other agents that either enhance the activity of the inhibitor or compliment its activity or use in treatment. Such additional factors and/or agents may produce an augmented or even synergistic effect when administered with a PI3Kδ selective inhibitor, or minimize side effects. In one embodiment, the methods of the invention may include administering formulations comprising a PI3Kδ selective inhibitor of the invention with a particular cytokine, lymphokine, other hematopoietic factor, thrombolytic or anti-thrombotic factor, or anti-inflammatory agent before, during, or after administration of the PI3Kδ selective inhibitor. Many cytokines, lymphokines, hematopoietic factors, thrombolytic or anti-thrombotic factors, and anti-inflammatory agents act in a proangiogenic manner in the presence of angiogenic regulators including but not limited to VEGF, and in an anti-angiogenic manner in the absence of such positive angiogenic regulators. Additionally, the activity of such ‘dualistic’ agents may depend on the targeted tissue type and/or stage of development. Nonetheless, one of ordinary skill can easily determine if a particular cytokine, lymphokine, hematopoietic factor, thrombolytic or anti-thrombotic factor, and/or anti-inflammatory agent enhances or compliments the activity or use of the PI3Kδ selective inhibitors in treatment.

More specifically, and without limitation, the methods of the invention may comprise administering a PI3Kδ selective inhibitor with one or more of TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Pharmaceutical compositions in accordance with the invention may also include other known angiopoietins such as Ang-2, Ang-4, and Ang-Y, growth factors such as bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

Additionally, and without limitation, the methods of the invention may comprise administering a PI3Kβ selective inhibitor with one or more chemotherapeutic agents including but not limited to alkylating agents, intercalating agents, antimetabolites, natural products, biological response modifiers, miscellaneous agents, and hormones and antagonists. Alkylating agents for use in the inventive methods include but are not limited to nitrogen mustards such as mechlorethamine, cyclophosphamide, ifosfamide, melphalan and chlorambucil, nitrosoureas such as carmustine (BCNU), lomustine (CCNU) and semustine (methyl-CCNU), ethylenimine/methylmelamines such as triethylenemelamine (TEM), triethylene thiophosphoramide (thiotepa) and hexamethylmelamine (HMM, altretamine), alkyl sulfonates such as busulfan, and triazines such as dacarbazine (DTIC). Antimetabolites include but are not limited to folic acid analogs (including methotrexate and trimetrexate), pyrimidine analogs (including 5-fluorouracil, fluorodeoxyuridine, gemcitabine, cytosine arabinoside (AraC, cytarabine), 5-azacytidine and 2,2*-difluorodeoxycytidine), and purine analogs (including 6-mercaptopurine, 6-thioguanine, azathioprine, 2′-deoxycoformycin (pentostatin), erythrohydroxynonyladenine (EHNA), fludarabine phosphate and 2-chlorodeoxyadenosine (cladribine, 2-CdA)). Intercalating agents for use in the inventive methods include but are not limited to ethidium bromide and acridine. Natural products for use in the inventive methods include but are not limited to anti-mitotic drugs such as paclitaxel, docetaxel, vinca alkaloids (including vinblastine (VLB), vincristine, vindesine and vinorelbine), taxotere, estramustine and estramustine phosphate. Additional natural products for use in the inventive methods include epipodophyllotoxins such as etoposide and teniposide, antibiotics such as actimomycin D, daunomycin (rubidomycin), doxorubicin, mitoxantrone, idarubicin, bleomycins, plicamycin (mithramycin), mitomycin C, dactinomycin and actinomycin D, and enzymes such as L-asparaginase. Biological response modifiers for use in the inventive methods include but are not limited to interferon-alpha, IL-2, G-CSF and GM-CSF. Miscellaneous agents for use in the inventive methods include but are not limited to platinum coordination complexes such as cisplatin and carboplatin, anthracenediones such as mitoxantrone, substituted ureas such as hydroxyurea, methylhydrazine derivatives such as N-methylhydrazine (MIH) and procarbazine, and adrenocortical suppressants such as mitotane (o,p*-DDD) and aminoglutethimide. Hormones and antagonists for use in the inventive methods include but are not limited to adrenocorticosteroids/antagonists such as prednisone, dexamethasone and aminoglutethimide, progestins such as hydroxyprogesterone caproate, medroxyprogesterone acetate and megestrol acetate, estrogens such as diethylstilbestrol and ethinyl estradiol, antiestrogens such as tamoxifen, androgens such as testosterone propionate and fluoxymesterone, antiandrogens such as flutamide, gonadotropin-releasing hormone analogs and leuprolide, and non-steroidal antiandrogens such as flutamide.

In one aspect, the chemotherapeutic is a DNA-damaging chemotherapeutic. Specific types of DNA-damaging chemotherapeutic agents contemplated for use in the inventive methods include, e.g., alkylating agents and intercalating agents.

The methods of the invention can also further comprise administering a PI3Kδ selective inhibitor in combination with a photodynamic therapy protocol. Typically, a photosensitizer is administered orally, intravenously, or topically, and then activated by an external light source. Photosensitizers for use in the methods of the invention include but are not limited to psoralens, lutetium texaphyrin (Lutex), benzoporphyrin derivatives (BPD) such as Verteporfin and Photofrin porfimer sodium (PH), phthalocyanines and derivatives thereof. Lasers are typically used to activate the photosensitizer. Light-emitting diodes (LEDs) and florescent light sources can also be used, but these do result in longer treatment times.

Additionally, and without limitation, the methods of the invention may comprise administering a PI3Kδ selective inhibitor with one or more additional anti-angiogenic agents including but not limited to plasminogen fragments such as angiostatin and endostatin; angiostatic steroids such as squalamine; matrix metalloproteinase inhibitors such as Bay-129566; anti-vascular endothelial growth factor (anti-VEGF) isoform antibodies; anti-VEGF receptor antibodies; inhibitors that target VEGF isoforms and their receptors; inhibitors of growth factor (e.g., VEGF, PDGF, FGF) receptor tyrosine kinase catalytic activity such as SU11248; inhibitors of FGF production such as interferon alpha; inhibitors of methionine aminopeptidase-2 such as TNP-470; copper reduction therapies such as tetrathiomolybdate; inhibitors of FGF-triggered angiogenesis such as thalidomide and analogues thereof; platelet factor 4; and thrombospondin.

Methods of the invention contemplate use of PI3Kδ selective inhibitor compound having formula (I) or pharmaceutically acceptable salts and solvates thereof: 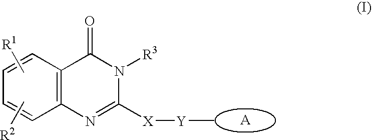
wherein A is an optionally substituted monocyclic or bicyclic ring system containing at least two nitrogen atoms, and at least one ring of the system is aromatic;

X is selected from the group consisting of C(R^{b})₂, CH₂CHR^{b}, and CH═C(R^{b});

Y is selected from the group consisting of null, S, SO, SO₂, NH, O, C(═O), OC(═O), C(═O)O, and NHC(═O)CH₂S;

R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

or R¹ and R² are taken together to form a 3- or 4-membered alkylene or alkenylene chain component of a 5- or 6-membered ring, optionally containing at least one heteroatom;

R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

As used herein, the term “alkyl” is defined as straight chained and branched hydrocarbon groups containing the indicated number of carbon atoms, typically methyl, ethyl, and straight chain and branched propyl and butyl groups. The hydrocarbon group can contain up to 16 carbon atoms, for example, one to eight carbon atoms. The term “alkyl” includes “bridged alkyl,” i.e., a C₆-C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except for containing a carbon-carbon double bond. “Cycloalkenyl” is defined similarly to cycloalkyl, except a carbon-carbon double bond is present in the ring.

The term “alkylene” is defined as an alkyl group having a substituent. For example, the term “C₁₋₃alkylenearyl” refers to an alkyl group containing one to three carbon atoms, and substituted with an aryl group.

The term “heteroC₁₋₃alkyl” is defined as a C₁₋₃alkyl group further containing a heteroatom selected from O, S, and NR^{a}. For example, —CH2OCH3 or —CH2CH2SCH3. The term “arylheteroC1-3alkyl” refers to an aryl group having a heteroC1-3alkyl substituent.

The term “halo” or “halogen” is defined herein to include fluorine, bromine, chlorine, and iodine.

The term “aryl,” alone or in combination, is defined herein as a monocyclic or polycyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, and amino. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like. The terms “arylC1-3alkyl” and “heteroarylC1-3alkyl” are defined as an aryl or heteroaryl group having a C1-3alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, and amino. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

Alternatively, the PI3Kδ selective inhibitor may be a compound having formula (II) or pharmaceutically acceptable salts and solvates thereof:

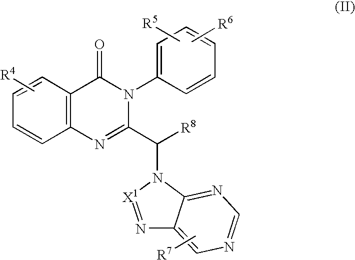

wherein R⁴, R⁵, R⁶, and R⁷, independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃ alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R⁸ is selected from the group consisting of hydrogen, C₁₋₆alkyl, halo, CN, C(═O)R^{a}, and C(═O)OR^{a};

X¹ is selected from the group consisting of CH (i.e., a carbon atom having a hydrogen atom attached thereto) and nitrogen;

Ra is selected from the group consisting of hydrogen, C₁₋₆alkyl, C₃₋₈cycloalkyl, C₃₋₈heterocycloalkyl, C₁₋₃alkyleneN(Rc)2, aryl, arylC₁₋₃alkyl, C₁₋₃alkylenearyl, heteroaryl, heteroarylC₁₋₃alkyl, and C₁₋₃alkyleneheteroaryl;

or two Ra groups are taken together to form a 5- or 6-membered ring, optionally containing at least one heteroatom;

Rc is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

The PI3Kδ selective inhibitor may also be a compound having formula (III) or pharmaceutically acceptable salts and solvates thereof:

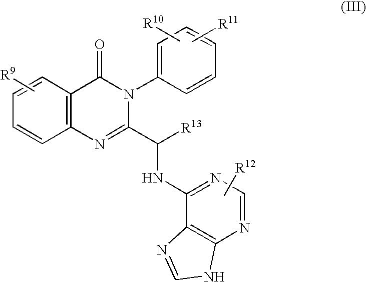

wherein R⁹, R¹⁰, R¹¹, and R¹², independently, are selected from the group consisting of hydrogen, amino, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R¹³ is selected from the group consisting of hydrogen, C₁₋₆alkyl, halo, CN, C(═O)R^{a}, and C(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

More specifically, representative PI3Kδ selective inhibitors in accordance with the foregoing chemical formulae include but are not limited to 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro4-oxo-3,4-dihydro-quinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}5-methyl-2-(9H-purin-6-ylsulfanylmethyl)-3H-quin-azolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3,5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimidin-3-yl)-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 5-methyl-3-phenyl-2-[1-(9H-purin-6-ylamino)-propyl]-3H-quinazolin-4-one; 2-[1-(2-fluoro-9H-purin-6-ylamino)-ethyl]-5-methyl-3-phenyl-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-difluoro-phenyl)-5-methyl-3H-quinazolin-4-one; 3-(2,6-difluoro-phenyl)-2-[1-(2-fluoro-9H-purin-6-ylamino)-ethyl]-5-methyl-3H-quinazolin-4-one; 3-(2,6-difluoro-phenyl)-5-methyl-2-[1-(7H-pyrrolo[2,3-d]pyrimidin-4-ylamino)-ethyl]-3H-quinazolin-4-one; 2-[1-(2-amino-9H-purin-6-ylamino)-propyl]-5-methyl-3-phenyl-3H-quinazolin-4-one; 5-methyl-3-phenyl-2-[1-(7H-pyrrolo[2,3-d]pyrimidin-4-ylamino)-propyl]-3H-quinazolin-4-one; 2-[1-(2-fluoro-9h-purin-6-ylamino)-propyl]-5-methyl-3-phenyl-3h-quinazolin-4-one; 5-methyl-3-phenyl-2-[1-(9H-purin-6-ylamino)-ethyl]-3H-quinazolin-4-one; 2-[1-(2-amino-9H-purin-6-ylamino)-ethyl]-5-methyl-3-phenyl-3H-quinazolin-4-one; 2-[2-benzyloxy-1-(9H-purin-6-ylamino)-ethyl]-5-methyl-3-phenyl-3H-quinazolin-4-one; 2-[1-(2-amino-9H-purin-6-ylamino)-2-benzyloxy-ethyl]-5-methyl-3-phenyl-3H-quinazolin-4-one; 2-[2-benzyloxy-1-(7H-pyrrolo[2,3-d]pyrimidin-4-ylamino)-ethyl]-5-methyl-3-phenyl-3H-quinazolin-4-one; 2-[2-benzyloxy-1-(2-fluoro-9H-purin-6-ylamino)-ethyl]-5-methyl-3-phenyl-3H-quinazolin-4-one; 3-(4-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(4-fluoro-phenyl)-5-methyl-3H-quinazolin-4-one; 3-(4-fluoro-phenyl)-2-[1-(2-fluoro-9H-purin-6-ylamino)-ethyl]-5-methyl-3H-quinazolin-4-one; 3-(4-fluoro-phenyl)-5-methyl-2-[1-( 7H-pyrrolo[2,3-d]pyrimidin-4-ylamino)-ethyl]-3H-quinazolin-4-one; 5-methyl-3-phenyl-2-[1-(7H-pyrrolo[2,3-d]pyrimidin-4-ylamino)-ethyl]-3H-quinazolin-4-one; 3-(3-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-fluoro-phenyl)-5-methyl-3H-quinazolin-4-one; 3-(3-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(9H-purin-6-yl)-pyrrolidin-2-yl]-3H-quinazolin-4-one; 2-[2-hydroxy-1-(9H-purin-6-ylamino)-ethyl]-5-methyl-3-phenyl-3H-quinazolin-4-one; 5-methyl-3-phenyl-2-[phenyl-(9H-purin-6-ylamino)-methyl]-3H-quinazolin-4-one; 2-[(2-amino-9H-purin-6-ylamino)-phenyl-methyl]-5-methyl-3-phenyl-3H-quinazolin-4-one; 2-[(2-fluoro-9H-purin-6-ylamino)-phenyl-methyl]-5-methyl-3-phenyl-3H-quinazolin-4-one; 5-methyl-3-phenyl-2-[phenyl-(7H-pyrrolo[2,3-d]pyrimidin-4-ylamino)-methyl]-3H-quinazolin-4-one; 5-fluoro-3-phenyl-2-[1-(9H-purin-6-ylamino)-ethyl]-3H-quinazolin-4-one; 2-[1-(2-amino-9H-purin-6-ylamino)-ethyl]-5-fluoro-3-phenyl-3H-quinazolin-4-one; 2-[1-(2-amino-9H-purin-6-ylamino)-ethyl]-5-chloro-3-phenyl-3H-quinazolin-4-one; [5-(5-methyl-4-oxo-3-phenyl-3,4-dihydro-quinazolin-2-yl)-5-(9H-purin-6-ylamino)-pentyl]-carbamic acid benzyl ester; [5-(2-amino-9H-purin-6-ylamino)-5-(5-methyl-4-oxo-3-phenyl-3,4-dihydro-quinazolin-2-yl)-pentyl]-carbamic acid benzyl ester; [4-(5-methyl-4-oxo-3-phenyl-3,4-dihydro-quinazolin-2-yl)-4-(9H-purin-6-ylamino)-butyl]-carbamic acid benzyl ester; [4-(2-amino-9H-purin-6-ylamino)-4-(5-methyl-4-oxo-3-phenyl-3,4-dihydro-quinazolin-2-yl)-butyl]-carbamic acid benzyl ester; 3-phenyl-2-[1-(9H-purin-6-ylamino)-ethyl]-3H-quinazolin-4-one; 2-[5-amino-1-(9H-purin-6-ylamino)-pentyl]-5-methyl-3-phenyl-3H-quinazolin-4-one); 2-[5-amino-1-(2-amino-9H-purin-6-ylamino)-pentyl]-5-methyl-3-phenyl-3H-quinazolin-4-one; 2-[1-(2-amino-9H-purin-6-ylamino)-ethyl]-3-(2,6-Dimethyl-phenyl)-5-methyl-3H-quinazolin-4-one; 3-(2,6-dimethyl-phenyl)-5-methyl-2-[1-(9H-purin-6-ylamino)-ethyl]-3H-quinazolin-4-one; 5-morpholin-4-ylmethyl-3-phenyl-2-[1-(9H-purin-6-ylamino)-ethyl]-3H-quinazolin-4-one; 2-[1-(2-amino-9H-purin-6-ylamino)-ethyl]-5-morpholin-4-ylmethyl-3-phenyl-3H-quinazolin-4-one; 2-[4-amino-1-(2-amino-9H-purin-6-ylamino)-butyl]-5-methyl-3-phenyl-3H-quinazolin-4-one; 6-fluoro-3-phenyl-2-[1-(9H-purin-6-ylamino)-ethyl]-3H-quinazolin-4-one; 2-[1-(2-amino-9H-purin-6-ylamino)-ethyl]-6-fluoro-3-phenyl-3H-quinazolin-4-one; 2-[2-tert-butoxy-1-(9H-purin-6-ylamino)-ethyl]-5-methyl-3-phenyl-3H-quinazolin-4-one; 3-(3-methyl-phenyl)-5-methyl-2-[1-(9H-purin-6-ylamino)-ethyl]-3H-quinazolin-4-one; 2-[1-(2-amino-9H-purin-6-ylamino)-ethyl]-3-(3-methyl-phenyl)-5-methyl-3H-quinazolin-4-one; 3-(3-chloro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-chloro-phenyl)-5-methyl-3H-quinazolin-4-one; 2-[1-(2-amino-9H-purin-6-ylamino)-2-hydroxy-ethyl]-5-methyl-3-phenyl-3H-quinazolin-4-one; 2-[1-(2-amino-9H-purin-6-ylamino)-ethyl]-3-(3-fluoro-phenyl)-3H-quinazolin-4-one; 2-[1-(2-amino-9H-purin-6-ylamino)-ethyl]-3-(2,6-difluoro-phenyl)-3H-quinazolin-4-one; 2-[1-(2-amino-9H-purin-6-ylamino)-propyl]-5-fluoro-3-phenyl-3H-quinazolin-4-one; 5-chloro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 3-phenyl-2-[1-(9H-purin-6-ylamino)-ethyl]-5-trifluoromethyl-3H-quinazolin-4-one; 3-(2,6-difluoro-phenyl)-5-methyl-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-propyl]-3-(2,6-difluoro-phenyl)-5-methyl-3H-quinazolin-4-one; 2-[1-(2-amino-9H-purin-6-ylamino)-ethyl]-3-(2,6-difluoro-phenyl)-5-methyl-3H-quinazolin-4-one; 3-(3,5-dichloro-phenyl)-5-methyl-2-[1-(9H-purin-6-ylamino)-ethyl]-3H-quinazolin-4-one; 3-(2,6-dichloro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-dichloro-phenyl)-5-methyl-3H-quinazolin-4-one; 5-chloro-3-phenyl-2-[1-(9H-purin-6-ylamino)-propyl]-3H-quinazolin-4-one; 2-[1-(2-amino-9H-purin-6-ylamino)-propyl]-5-chloro-3-phenyl-3H-quinazolin-4-one; 5-methyl-3-phenyl-2-[1-(9H-purin-6-ylamino)-butyl]-3H-quinazolin-4-one; 2-[1-(2-amino-9H-purin-6-ylamino)-butyl]-5-methyl-3-phenyl-3H-quinazolin-4-one; 2-[1-(2-amino-9H-purin-6-ylamino)-ethyl]-3-(3,5-dichloro-phenyl)-5-methyl-3H-quinazolin-4-one; 5-methyl-3-(3-morpholin-4-ylmethyl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-ylmethyl-phenyl)-3H-quinazolin-4-one; 2-[1-(5-bromo-7H-pyrrolo[2,3-d]pyrimidin-4-ylamino)-ethyl]-5-methyl-3-phenyl-3H-quinazolin-4-one; 5-methyl-2-[1-(5-methyl-7H-pyrrolo[2,3-d]pyrimidin-4-ylamino)-ethyl]-3-phenyl-3H-quinazolin-4-one; 2-[1-(5-fluoro-7H-pyrrolo[2,3-d]pyrimidin-4-ylamino)-ethyl]-5-methyl-3-phenyl-3H-quinazolin-4-one; 2-[2-hydroxy-1-(9H-purin-6-ylamino)-ethyl]-3-phenyl-3H-quinazolin-4-one; 3-(3,5-difluoro-phenyl)-5-methyl-2-[1-(9H-purin-6-ylamino)-propyl]-3H-quinazolin-4-one; 2-[1-(2-amino-9H-purin-6-ylamino)-propyl]-3-(3,5-difluoro-phenyl)-5-methyl-3H-quinazolin-4-one; 3-(3,5-difluoro-phenyl)-2-[1-(9H-purin-6-ylamino)-ethyl]-3H-quinazolin-4-one; 2-[1-(5-bromo-7H-pyrrolo[2,3-d]pyrimidin-4-ylamino)-ethyl]-3-(3-fluoro-phenyl)-5-methyl-3H-quinazolin-4-one; 3-(3-fluoro-phenyl)-5-methyl-2-[1-(5-methyl-7H-pyrrolo[2,3-dipyrimidin-4-ylamino)-ethyl]-3H-quinazolin-4-one; 3-phenyl-2-[1-(9H-purin-6-ylamino)-propyl]-3H-quinazolin-4-one; 2-[1-(2-amino-9H-purin-6-ylamino)-ethyl]-3-(3,5-difluoro-phenyl)-3H-quinazolin-4-one; 2-[1-(2-amino-9H-purin-6-ylamino)-propyl]-3-phenyl-3H-quinazolin-4-one; 6,7-difluoro-3-phenyl-2-[-1-(9H-purin-6-ylamino)-ethyl]-3H-quinazolin-4-one; 6-fluoro-3-(3-fluoro-phenyl)-2-[1-(9H-purin-6-ylamino)-ethyl]-3H-quinazolin-4-one; 2-[4-diethylamino-1-(9H-purin-6-ylamino)-butyl]-5-methyl-3-phenyl-3H-quinazolin-4-one; 5-fluoro-3-phenyl-2-[1-(9H-purin-6-ylamino)-propyl]-3H-quinazolin-4-one; 3-phenyl-2-[1-(9H-purin-6-ylamino)-ethyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-2-[1-(2-fluoro-9H-purin-6-ylamino)-ethyl]-3-phenyl-3H-quinazolin-4-one; 3-(3-fluoro-phenyl)-2-[1-(9H-purin-6-ylamino)-ethyl]-3H-quinazolin-4-one; 5-chloro-3-(3,5-difluoro-phenyl)-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 3-(2,6-difluoro-phenyl)-2-[1-(9H-purin-6-ylamino)-ethyl]-3H-quinazolin-4-one; 5-Methyl-3-phenyl-2-[3,3,3-trifluoro-1-(9H-purin-6-ylamino)-propyl]-3H-quinazolin-4-one; 3-(3-hydroxy-phenyl)-5-methyl-2-[1-(9H-purin-6-ylamino)-ethyl]-3H-quinazolin-4-one; 3-(3-methoxy-phenyl)-5-methyl-2-{1-[9H-purin-6-ylamino]-ethyl}3H-quinazolin-4-one; 3-[3-(2-dimethylamino-ethoxy)-phenyl]-5-methyl-2-{1-[9H-purin-6-ylamino]-ethyl}-3H-quinazolin-4-one; 3-(3-cyclopropylmethoxy-phenyl)-5-methyl-2-{1-[9H-purin-6-ylamino]-ethyl}-3H-quinazolin-4-one; 5-methyl-3-(3-prop-2-ynyloxy-phenyl)-2-{1-[9H-purin-6-ylamino]-ethyl}3H-quinazolin-4-one; 2-{1-[2-amino-9H-purin-6-ylamino]ethyl}3-(3-hydroxyphenyl)-5-methyl-3H-quinazolin-4-one; 2-{1-[2-amino-9H-purin-6-ylamino]ethyl}3-(3-methoxyphenyl)-5-methyl-3H-quinazolin-4-one; 2-{1-[2-amino-9H-purin-6-ylamino]ethyl}-3-(3-cyclopropylmethoxy-phenyl)-5-methyl-3H-quinazolin-4-one; 2-{1-[2-amino-9H-purin-6-ylamino]ethyl}-5-methyl-3-(3-prop-2-ynyloxy-phenyl)-3H-quinazolin-4-one; 3-(3-ethynyl-phenyl)-5-methyl-2-[1-(9H-purin-6-ylamino)-ethyl]-3H-quinazolin-4-one; 3-{5-methyl-4-oxo-2-[1-(9H-purin-6-ylamino)-ethyl]-4H-quinazolin-3-yl}-benzonitrile; 3-{5-methyl-4-oxo-2-{1-[9H-purin-6-ylamino)-ethyl]-4H-quinazolin-3-yl}-benzamide; 3-(3-acetyl-phenyl)-5-methyl-2-{1-[9H-purin-6-ylamino]-ethyl}-3H-quinazolin-4-one; 2-(3-(5-methyl-4-oxo-2-{1-[9H-purin-6-ylamino]-ethyl}-4H-quinazolin-3-yl-phenoxy acetamide; 5-methyl-2-{1-[9H-purin-6-ylamino]-ethyl}-3-[3-(tetrahydropuran-4-yloxy)-phenyl]-3H-quinazolin-4-one; 3-[3-(2-methoxy-ethoxy)-phenyl]-5-methyl-2-[1-(9H-purin-6-ylamino)-ethyl]-3H-quinazolin-4-one; 6-fluoro-2-[1-(9H-purin-6-ylamino)ethyl]-3-[3-(tetrahydro-pyran-4-yloxy)-phenyl]-3H-quinazolin-4-one; 3-[3-(3-dimethylamino-propoxy)-phenyl]-5-methyl-2-[1-(9H-purin-6-ylamino)-ethyl]-3H-quinazolin-4-one; 2-[1-(2-amino-9H-purin-6-ylamino)-ethyl]-3-(3-ethynyl-phenyl)-5-methyl-3H-quinazolin-4-one; 3-{2-[1-(2-amino-9H-purin-6-ylamino)-ethyl]-5-methyl-4-oxo-4H-quinazolin-3-yl}-benzonitrile; 3-{2-[1-(2-amino-9H-purin-6-ylamino)-ethyl]-5-methyl-4-oxo-4H-quinazolin-3-yl}benzamide; 3-{2-[1-(2-amino-9H-purin-6-ylamino)-ethyl]-5-methyl-4-oxo4H-quinazolin-3-yl}-benzamide; 5-methyl-3-(3-morpholin-4-yl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-yl-phenyl)-3H-quinazolin-4-one; 2-[1-(2-amino-9H-purin-6-ylamino)-ethyl]-3-[3-(2-methoxy-ethoxy)-phenyl]-5-methyl-3H-quinazolin-4-one; 2-[1-(2-amino-9H-purin-6-ylamino)-ethyl]-3-[3-(2-dimethylamino-ethoxy)-phenyl]-5-methyl-3H-quinazolin-4-one; 2-[1-(2-amino-9H-purin-6-ylamino)-but-3-ynyl]-5-methyl-3-phenyl-3H-quinazolin-4-one; 2-[1-(2-amino-9H-purin-6-ylamino)-but-3-ynyl]-5-methyl-3-phenyl-3H-quinazolin-4-one; 5-chloro-3-(3, 5-difluoro-phenyl)-2-[1-(9H-purin-6-ylamino)-ethyl]-3H-quinazolin-4-one; 2-[1-(2-amino-9H-purin-6-ylamino)-propyl]-5-chloro-3-(3,5-difluoro-phenyl)-3H-quinazolin-4-one; 2-[1-(2-amino-9H-purin-6-ylamino)-ethyl]-5-chloro-3-(3,5-difluoro-phenyl)-3H-quinazolin-4-one; 3-(3,5-difluoro-phenyl)-6-fluoro-2-[1-(9H-purin-6-ylamino)-ethyl]-3H-quinazolin-4-one; 5-chloro-3-(2,6-difluoro-phenyl)-2-[1-(9H-purin-6-ylamino)-propyl]-3H-quinazolin-4-one; 2-[1-(2-amino-9H-purin-6-ylamino)-propyl]-5-chloro-3-(2,6-difluoro-phenyl)-3H-quinazolin-4-one; 5-methyl-3-phenyl-2-[1-(9H-purin-6-yloxy)-ethyl]-3H-quinazolin-4-one; and mixtures thereof.

Where a stereocenter is present, the methods can be practiced using a racemic mixture of the compounds or a specific enantiomer. In preferred embodiments where a stereocenter is present, the S-enantiomer of the above compounds is utilized. However, the methods of the invention include administration of all possible stereoisomers and geometric isomers of the aforementioned compounds.

Additionally, the methods include administration of PI3Kδ selective inhibitors comprising an arylmorpholine moiety [Knight et al., Bioorganic & Medicinal Chemistry, 12:4749-4759 (2004)]. Representative PI3Kδ selective inhibitors include but are not limited to 2-morpholin-4-yl-8-o-tolyloxy-1H-quinolin-4-one; 9-bromo-7-methyl-2-morpholin-4-yl-pyrido(1,2-a)-pyrimidin-4-one; 9-benzylamino-7-methyl-2-morpholin-4-yl-pyrido-(1,2a)pyrimidin-4-one; 9-(3-amino-phenyl)-7-methyl-2-morpholin-4-yl-pyrido[1,2-a]pyrimidin-4-one; 9-(2-methoxy-phenylamino)-7-methyl-2-morpholin-4-yl-pyrido(1,2-a)pyrimidin-4-one; 7-methyl-2-morpholin-4-yl-9-o-tolylamino-pyri-do(1,2-a)pyrimidin-4-one; 9-(3,4-dimethyl-phenylamino)-7-methyl-2-morph-olin-4-yl-pyrido(1,2-a)pyrimidin-one; 7-methyl-9-(3-methyl-benzylamino)-2-morpholin-4-yl-pyrido(1,2-a)pyrimidin-4-one; 9-(2,3-dimethyl-phenylamino)-7-methyl-2-morpholin-4-yl-pyrido(1,2-a)pyrimidin-4-one; 7-methyl-9-(2-methyl-benzylamino)-2-morpholin-4-yl-pyrido(1,2-a) pyrimidin-4-one; 5-morpholin-4-yl-2-nitro-phenylamine; 1-(2-hydroxy-4-morpholin-4-yl-phenyl)-phenyl-methanone; and, 2-chloro-1-(2-hydroxy-4-morpholin-4-yl-phenyl)-ethanone.

Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma et al. (Eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Additionally, compounds that selectively negatively regulate p110δ mRNA expression more effectively than they do other isozymes of the PI3K family, and that possess acceptable pharmacological properties are contemplated for use as PI3Kδ selective inhibitors in the methods of the invention. Polynucleotides encoding human p110δ are disclosed, for example, in Genbank Accession Nos. AR255866, NM 005026, U86453, U57843 and Y10055, the entire disclosures of which are incorporated herein by reference [see also, Vanhaesebroeck et al., P.N.A.S., 94:4330-4335 (1997), the entire disclosure of which is incorporated herein by reference]. Representative polynucleotides encoding mouse p110δ are disclosed, for example, in Genbank Accession Nos. BC035203, AK040867, U86587, and NM_{—}008840, and a polynucleotide encoding rat p110δ is disclosed in Genbank Accession No. XM_{—}345606, in each case the entire disclosures of which are incorporated herein by reference.

In one embodiment, the invention provides methods using antisense oligonucleotides which negatively regulate p110δ expression via hybridization, to messenger RNA (mRNA) encoding p110δ. In one aspect, antisense oligonucleotides at least 5 to about 50 nucleotides in length, including all lengths (measured in number of nucleotides) in between, which specifically hybridize to mRNA encoding p110δ and inhibit mRNA expression, and as a result p110δ protein expression, are contemplated for use in the methods of the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides that are not perfectly complementary, i.e., those which include a limited number of mismatches with respect to a region in the target polynucleotide, also retain high degrees of hybridization specificity and therefore also can inhibit expression of the target mRNA. Accordingly, the invention contemplates methods using antisense oligonucleotides that are perfectly complementary to a target region in a polynucleotide encoding p110δ, as well as methods that utilize antisense oligonucleotides that are not perfectly complementary (i.e., include mismatches) to a target region in the target polynucleotide to the extent that the mismatches do not preclude specific hybridization to the target region in the target polynucleotide. Preparation and use of antisense compounds is described, for example, in U.S. Pat. No. 6,277,981, the entire disclosure of which is incorporated herein by reference [see also, Gibson (Ed.), Antisense and Ribozyme Methodology, (1997), the entire disclosure of which is incorporated herein by reference].

The invention further contemplates methods utilizing ribozyme inhibitors which, as is known in the art, include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The methods of the invention therefore contemplate ribozyme inhibitors comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target p110δ-encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250, 6,410,224, 5,225,347, the entiredisclosures of which are incorporated herein by reference.

The invention also contemplates use of methods in which RNAi technology is utilized for inhibiting p110δ expression. In one aspect, the invention provides double-stranded RNA (dsRNA) wherein one strand is complementary to a target region in a target p110δ-encoding polynucleotide. In general, dsRNA molecules of this type are less than 30 nucleotides in length and referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target p110δ-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. Patent Application No. 20040023390, the entire disclosure of which is incorporated herein by reference.

The invention further contemplates methods wherein inhibition of p110δ is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target p110δ-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the entire disclosure of which is incorporated herein by reference.

The invention also contemplates methods wherein inhibition of p110δ is effected using aptamers. Aptamers are highly selective nucleic acid based drugs (i.e., synthetic oligonucleotides) that are capable of bonding to specific target proteins, and thereby inhibiting target function. A typical aptamer is 10-15 kDa in size (approximately 30-45 nucleotides). Thus, aptamers advantageously work against both intra- and extra-cellular targets. The pharmacokinetics and biodistribution of aptamers have been generally described [Healy et al., Pharma. Res., 21:2234-2246 (2004)].

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

Derivatization with bifunctional agents is useful for cross-linking a compound of the invention to a support matrix or to a carrier. One such carrier is polyethylene glycol (PEG). The PEG group may be of any convenient molecular weight and may be straight chain or branched. The average molecular weight of the PEG can range from about 2 kDa to about 100 kDa, in another aspect from about 5 kDa to about 50 kDa, and in a further aspect from about 5 kDa to about 10 kDa. The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, ci-haloacetyl, maleimido or hydrazino group) to a reactive group on the target inhibitor compound (e.g., an aldehyde, amino, ester, thiol, α-haloacetyl, maleimido or hydrazino group). Cross-linking agents can include, e.g., esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for inhibitor immobilization.

The pharmaceutical compositions of the invention may also include compounds derivatized to include one or more antibody Fc regions. Fc regions of antibodies comprise monomeric polypeptides that may be in dimeric or multimeric forms linked by disulfide bonds or by non-covalent association. The number of intermolecular disulfide bonds between monomeric subunits of Fc molecules can be from one to four depending on the class (e.g., IgG, IgA, IgE) or subclass (e.g., IgG1, IgG2, IgG3, IgA1, IgGA2) of antibody from which the Fc region is derived. The term “Fc” as used herein is generic to the monomeric, dimeric, and multimeric forms of Fc molecules, with the Fc region being a wild type structure or a derivatized structure. The pharmaceutical compositions of the invention may also include the salvage receptor binding domain of an Fc molecule as described in WO 96/32478, as well as other Fc molecules described in WO 97/34631.

Such derivatized moieties preferably improve one or more characteristics of the inhibitor compounds of the invention, including for example, biological activity, solubility, absorption, biological half life, and the like. Alternatively, derivatized moieties result in compounds that have the same, or essentially the same, characteristics and/or properties of the compound that is not derivatized. The moieties may alternatively eliminate or attenuate any undesirable side effect of the compounds and the like.

Methods include administration of an inhibitor to an individual in need, by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Some commercially available diluents are Fast-Flo, Emdex, STA-Rx 1500, Emcompress and Avicell. Such compositions may influence the physical state, stability, rate of in vivo release, and rate of in vivo clearance of the PI3Kδ inhibitor compounds [see, e.g., Remington's Pharmaceutical Sciences, 18th Ed. pp. 1435-1712 (1990), which is incorporated herein by reference].

Pharmaceutically acceptable fillers can include, for example, lactose, microcrystalline cellulose, dicalcium phosphate, tricalcium phosphate, calcium sulfate, dextrose, mannitol, and/or sucrose.

Inorganic salts including calcium triphosphate, magnesium carbonate, and sodium chloride may also be used as fillers in the pharmaceutical compositions. Amino acids may be used such as use in a buffer formulation of the pharmaceutical compositions.

Disintegrants may be included in solid dosage formulations of the inhibitors. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the formulation of the therapeutic ingredient to prevent sticking during the formulation process. Lubricants may be used as a layer between the therapeutic ingredient and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the therapeutic ingredient during formulation and to aid rearrangement during compression might be added. Suitable glidants include starch, talc, pyrogenic silica and hydrated silicoaluminate.

To aid dissolution of the therapeutic into the aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulation may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

In the methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

Also contemplated herein is pulmonary delivery of the PI3Kδ inhibitors in accordance with the invention. According to this aspect of the invention, the inhibitor is delivered to the lungs of a mammal while inhaling and traverses across the lung epithelial lining to the blood stream.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn H nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

When used in pulmonary administration methods, the inhibitors of the invention are most advantageously prepared in particulate form with an average particle size of less than 10 μm (or microns), for example, 0.5 to 5μm, for most effective delivery to the distal lung.

Formulations suitable for use with a nebulizer, either jet or ultrasonic, will typically comprise the inventive compound dissolved in water at a concentration range of about 0.1 to 100 mg of inhibitor per mL of solution, 1 to 50 mg of inhibitor per mL of solution, or 5 to 25 mg of inhibitor per mL of solution. The formulation may also include a buffer. The nebulizer formulation may also contain a surfactant, to reduce or prevent surface induced aggregation of the inhibitor caused by atomization of the solution in forming the aerosol.

Formulations for use with a metered-dose inhaler device will generally comprise a finely divided powder containing the inventive inhibitors suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent or diluent such as lactose, sorbitol, sucrose, mannitol, trehalose, or xylitol in amounts which facilitate dispersal of the powder from the device, e.g., 50 to 90% by weight of the formulation.

Nasal delivery of the inventive compound is also contemplated. Nasal delivery allows the passage of the inhibitor to the blood stream directly after administering the therapeutic product to the nose, without the necessity for deposition of the product in the lung. Formulations for nasal delivery may include dextran or cyclodextran. Delivery via transport across other mucous membranes is also contemplated.

Toxicity and therapeutic efficacy of the PI3Kδ selective compounds can be determined by standard pharmaceutical procedures in cell cultures or experimental animals, e.g., for determining the LD50 (the dose lethal to 50% of the population) and the ED50 (the dose therapeutically effective in 50% of the population). Additionally, this information can be determined in cell cultures or experimental animals additionally treated with other therapies including but not limited to radiation, chemotherapeutic agents, photodynamic therapies, radiofrequency ablation, anti-angiogenic agents, and combinations thereof.

In practice of the methods of the invention, the pharmaceutical compositions are generally provided in doses ranging from 1 pg compound/kg body weight to 1000 mg/kg, 0.1 mg/kg to 100 mg/kg, 0.1 mg/kg to 50 mg/kg, and 1 to 20 mg/kg, given in daily doses or in equivalent doses at longer or shorter intervals, e.g., every other day, twice weekly, weekly, or twice or three times daily. The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage [see, for example, Remington's Pharmaceutical Sciences, pp. 1435-1712, the disclosure of which is hereby incorporated by reference]. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various indications involving angiogenesis.

In the combination methods involving administration of radiation, external radiation is typically administered to an individual in an amount of about 1.8 Gy/day to about 3 Gy/day to a total dose of 30 to 70 Gy, with the total doses being administered over a period of about two to about seven weeks. Alternatively, brachytherapy is administered in an amount of about 40 Gy over about three days or about 5 Gy/day to a total amount of about 15-20 Gy.

### EXAMPLES

The following examples are provided to illustrate the invention, but are not intended to limit the scope thereof.

### Example 1

### P110δ is Expressed in Endothelial Cells

Western blot experiments were conducted to determine whether p110δ was expressed in endothelial cells.

To determine whether the p110δ isoform is present in endothelial cells, total protein was extracted from HUVECs and human microvascular endothelial cells (HMVECs), and Western immunoblots containing antibodies specific for the delta isoform were utilized. HUVEC and HMVEC cell lines (Clonetics, Calif.) were maintained in EBM-2 medium supplemented with EGM-2 MV Singlequots (BioWhittaker). Only fourth or fifth passage cells were used.

The Western blot analyses showed that the p110δ isoform is expressed in HUVEC and HMVEC cells.

### Example 2

### Administration of a PI3Kδ Selective Inhibitor Increases Apoptosis and Tumor Radiosensivity

To determine whether p110δ inhibition contributes to cell viability, apoptosis and clonogenic survival assays were conducted in HUVECs treated with a PI3Kδ selective inhibitor and/or radiation. Clonogenic assays were also performed to determine whether a PI3Kδ selective inhibitor enhances tumor radiosensitivity.

An Eldorado 8 Teletherapy Co-60 Unit (Atomic Energy of Canada Limited) was used to irradiate the endothelial cell cultures at a dose rate of 0.84 Gy/min. Delivered dose was verified by use of thermoluminescence detectors.

The number of cells undergoing apoptosis was quantified by microscopic analysis of apoptotic nuclei. Cells were fixed and stained with hematoxylin and eosin (“H&E”) 24 hours after treatment with 6 Gy radiation and/or 100 nM PI3Kδ selective inhibitor. Cells were then examined by light microscopy. For each treatment group, five high power fields (40× objective) were examined, and the number of apoptotic and total cells was determined. From these numbers, the percentage of apoptotic cells for each group was determined.

The number of cells undergoing apoptosis was also quantified using an Annexin V-fluorescein (FITC) apoptosis assay and flow cytometry, as previously described [Vermes et al., J. Immunol. Meth., 184:39-51 (1995)]. If Annexin-V binds to a cell surface, cell death is imminent.

Clonogenic survival analysis was performed as previously described [Edwards et al., Cancer Res., 62:4671-77 (2002); Schueneman et al., Cancer Res., 63: 4009-4016 (2003)]. Briefly, HUVEC cultures were treated at radiation doses of 2 Gy, 4 Gy, and 6 Gy, with or without 100 nM PI3Kδ selective inhibitor for 30 minutes before irradiation. After treatment with radiation and/or 100 nM PI3Kδ selective inhibitor, cells were trypsinized, counted by hemocytometer, and subcultured into fresh medium. After 14 days, the cells were fixed with cold methanol and stained with 1% methylene blue. Colonies with at least 50 cells were counted, and the surviving fraction was determined.

The percentage of apoptotic endothelial cells was increased by 3.5% following treatment with radiation alone and by 3% following treatment with PI3Kδ selective inhibitor alone. When cells were pretreated with a PI3Kδ selective inhibitor and irradiation, a significant increase in apoptosis to 9% was observed (p=0.04). These data demonstrate a greater than additive effect of the combination of a PI3Kδ selective inhibitor and radiation as determined by multiplying the total amount of apoptosis achieved by each modality treatment individually to yield an expected value if the effects of each treatment modality were additive [see, e.g., Gorski et al., supra].

Treating the cells with a PI3Kδ selective inhibitor combined with radiation also significantly increased Annexin V staining as compared to either agent alone (p=0.02).

PI3Kδ selective inhibitor alone reduced plating efficiency to 90% as compared to untreated control cells, and in combination with 2 Gy increased cytotoxicity of endothelial cells by 10-fold. Clonogenic cell survival was significantly reduced when the cells were treated with PI3Kδ selective inhibitor prior to irradiation as compared to radiation alone (p=0.01). Accordingly, these data demonstrate that the radiosensitivity of cells treated with a combination therapy in accordance with the invention was significantly increased.

### Example 3

### Administration of a PI3Kδ Selective Inhibitor Increase Active Caspase-3 Levels in Endothelial Cells

Caspase-3 is a cysteine protease that promotes apoptotic cell death [Salvesen et al., Cell, 91:443-446 (1997)]. The protease is synthesized as an inactive 32 kDa pro-enzyme that can be converted by proteolysis to an active 17 kDa form [see, e.g., Stennicke et al., Biochim. Biophys. Acta. 1477(1-2):299-306 (2000); Kim et al., Endocrin., 141(5):1846-1853 (2000)]. Cell populations undergoing increased apoptosis produce higher amounts of the active form relative to cell populations undergoing apoptosis at a normal rate [see, e.g., Kim et al., supra]. Therefore, caspase-3 contents of HUVECS treated with a PI3Kδ selective inhibitor and/or radiation were measured to determine if inhibition of p110δ causes increased apoptosis.

The inactive and active caspase-3 forms can be differentiated and their contents measured by gel electrophoresis and protein blotting because of their different molecular mass. Pro-caspase-3 and active caspase-3 contents were determined for HUVECs at 6 and 24 hrs following treatment with either PI3Kδ selective inhibitor alone, 4 Gy radiation alone, PI3Kδ selective inhibitor alone, or a combination of 4 Gy and PI3Kδ selective inhibitor.

A significant increase in 17 kDa caspase-3 (active form) was observed with endothelial cells treated with PI3Kδ selective inhibitor alone and in combination with radiation. Therefore, treating endothelial cells with a PI3Kδ selective inhibitor alone and/or in combination with radiation increases apoptosis.

### Example 4

### Administration of a PI3Kδ Selective Inhibitor Attenuates Radiation Activation of AKT Phosphorylation

Radiation has previously been shown to induce the activation of Akt phosphorylation in a PI3K dependent manner [Edwards et al., supra]. To determine whether the p110δ isoform contributes to radiation-induced Akt phosphorylation, HUVEC cells were treated with a PI3Kδ selective inhibitor in accordance with the invention, with or without 3 Gy irradiation, and Akt phosphorylation was measured.

Cells were washed twice with phosphate buffer solution (PBS) and lysis buffer (20 nM Tris, 150 mM NaCl, 1 mM EDTA, 1% Triton X-100, 2.5 mM sodium pyrophosphate, 1 mM phenylmethylsulfonyl fluoride, and 1 μg/mL leupeptin) was added. Protein concentration was quantified by the BioRad method. 20 μg of total protein were loaded into each well and separated by 8% or 12% SDS-PAGE gel, depending on the size of the target protein being investigated. The proteins were transferred onto nitrocellulose membranes (Hybond ECL, Amersham, Arlington Heights, Ill.) and probed with antibodies for the phosphorylated Akt content and the total Akt content (New England Biolabs, Beverly, Mass.).

Following irradiation of the HUVEC cells (3 Gy), there was an increase in phosphorylation of Akt within 15 minutes of irradiation of the HUVEC cells. The administration of a PI3Kδ selective inhibitor in accordance with the invention was shown to attenuate radiation-induced Akt phosphorylation relative to HUVEC cells that did not receive PI3Kδ selective inhibitor (as measured by phosphorylated Akt content).

This example demonstrates that the administration of a PI3Kδ selective inhibitor to an individual receiving radiation therapy should facilitate/promote cellular apoptosis by reducing the amount of Akt phosphorylation induced by the radiation therapy. However, because the administration a PI3Kδ selective inhibitor reduces the amount of phosphorylated Akt, methods of administering such inhibitors are useful for treating individuals whether or not the individuals are additionally treated with other therapies.

### Example 5

### Administration of a PI3Kδ Selective Inhibitor Inhibits Tubule Formation in Endothelial Cells

Endothelial cells cultured in Matrigel™ form tubules within several hours. Endothelial cell tubule formation involves several physiologic processes including cytokinesia, intercellular signaling, and tubule differentiation. To determine whether inhibiting the activity of the p110δ isoform inhibits endothelial cell tubule formation, HUVEC cells were treated with a PI3Kδ selective inhibitor in accordance with the invention, with or without 3 Gy irradiation, and tubule formation was observed under microscope.

HUVEC cells were grown to about 80% confluence in 100 mm dishes. A PI3Kδ selective inhibitor in accordance with the invention (100 nM) was added to the cells for about 1 hour, and then the cells were treated with or without 3 Gy radiation. Subsequent to irradiation, the cells were washed with PBS twice, detached with 1% trypsin and 10⁵ cells were seeded per well onto wells coated with 200 μL of 10 mg/mL Matrigel™ solution (BD Bioscience, Bedford, Mass.) HUVECs medium (Iscove's modified Dulbecco's/Ham F-12 medium supplemented with 15% fetal calf serum, 1% penicillin-streptomycin, 45 μg of heparin per ml, and 10 μg of endothelial cell growth supplement per mL.). The plate was allowed to sit at room temperature for 15 minutes, and then incubated at 37° C. for 30 minutes to allow the Matrigel™ to polymerize. The cells were incubated for 24 hours to allow capillary-like tubule formation. Medium was removed carefully after incubation, and agarose was gently added to cells for optimal visualization. After solidification of agarose, immobilized tubes were fixed and stained with Diff-Quick solution. Stained tubules were washed 3× with PBS. The relative quantity of tubules was quantified by microscopic visualization and counting.

The administration of radiation alone (2 Gy) had no significant effect on HUVEC cell tubule formation in Matrigel™, whereas the administration of PI3Kδ selective inhibitor alone (100 nM) was shown to reduce tubule density by about 25% 48 hours after compound administration relative to a control. When the administration of a PI3Kδ selective inhibitor (100 nm) is combined with radiation (2 Gy), capillary-like tubule formation was almost completely eliminated and tubule formation was significantly attenuated (p=0.03).

These data demonstrate a synergistic or greater than additive effect of the combination of a PI3Kδ selective inhibitor and radiation as determined by multiplying the reduction in tubule formation achieved by each modality treatment individually to yield an expected value if the effects of each treatment modality were additive.

### Example 6

### Administration of a PI3Kδ Selective Inhibitor Inhibits Endothelial Cell Migration

The generation of new blood vessels involves multiple steps, including dissolution of the membrane of the originating vessel, endbthelial cell migration and proliferation, and formation of new vascular tubules [Ausprunk et al., supra]. Suppression of any one of these steps inhibits the formation of new blood vessels to the tumor and therefore affects tumor growth and metastasis. To determine whether the p110δ isoform contributes to endothelial cell migration, HUVEC cells were treated with a PI3Kδ selective inhibitor, with or without 3 Gy irradiation, in the presence of a growth factor that induces angiogenesis and thus endothelial cell migration.

HUVECs were grown to about 80% confluence in 100 mm dishes. The cells were subsequently washed two times with sterile PBS. Trypsin buffer was then added and the cells were incubated at about 37° C. for about 3 minutes. Trypsin digestion was then inhibited by the addition of complete growth medium. Approximately 2.5×10⁵ HUVEC cells were placed into a fibronectin-coated Boyden chamber in EGM-2 medium (Cambrex, East Rutherford, N.J.). A PI3Kδ selective inhibitor in accordance with the invention (100 nM) was added to the fibronectin-coated chamber.

The cells were treated with a PI3Kδ selective inhibitor with or without 3 Gy irradiation prior to plating on membrane. The cells were then incubated at about 37° C. for about 6 hours. Cells that did not migrate into the membrane and stay on upside of the membrane were removed by use of swabs. Media and cells were again swabbed from the inside of the chamber. Chambers were then placed into wells containing Cell Stain Solution (Chemicon International) and incubated for 30 minutes at room temperature. Cell stain was then removed from the wells and the cells were washed 3 times with PBS. The Boyden chambers were then washed with distilled water. Cells that migrated to the bottom of the membrane were counted by microscopy. Cell stain was then extracted by use of extraction buffer (Chemicon International) on a shaker for 5 to 10 minutes. 100 μl of stained solution from cell extractions was placed into a microtiter plate and absorbance was read at 550 nm.

VEGF was used as the growth factor in Boyden chamber migration assays. Bovine serum albumin (BSA) coated chambers served as negative controls.

Cells treated with a PI3Kδ selective inhibitor showed a reduction in cell migration as compared to untreated control cells. Cells treated with radiation alone showed an increased rate of migration as compared to untreated control cells. Additionally, cells treated with a PI3Kδ selective inhibitor and radiation showed a significant reduction in cell migration as compared with radiation alone (p=0.01).

Therefore, treating endothelial cells with a PI3Kδ selective inhibitor alone and/or in combination with radiation decreases endothelial cell migration.

### Example 7

### A Tumor Vascular Window Chamber Model Demonstrates the Efficacy of Administering a PI3Kδ Selective Inhibitor

To determine whether the administration of a PI3Kδ selective inhibitor in accordance with the invention enhances destruction of tumor vasculature, a PI3Kδ selective inhibitor was administered, with or without 2 Gy irradiation, to mice having implanted tumors. The tumor vascular linear density (VLD) was measured by use of an intravital tumor vascular window chamber. The time- and dose-dependent responses of tumor blood vessels were monitored.

Lewis Lung Carcinoma (LLC) cells were obtained from American Type Tissue Culture and were maintained in DMEM supplemented with 10% FCS and 1% penicillin-streptomycin. The cells were incubated in a 37° C. in a 5% CO₂ incubator. LLC tumors were established by injecting LLC cells into the C57BL6 mice prior to installation of the tumor vascular window chamber model.

The tumor vascular window chamber is a 3-g plastic frame that facilitates the viewing of an implanted tumor, and includes a bottom portion and a top portion. The intravital tumor vascular window chambers remained attached for the duration of the study. The window chambers were attached to the mice in accordance with the following protocol.

A penicillin-streptomycin solution (200 μL) was injected into the hind limb of a C57B6J mouse. A midline was found along the animal's back, and a clip was placed to hold the skin in position. A template, equivalent to the outer diameter of the window chamber, was traced, to give an incision outline. A circular incision was made tracing the perimeter (7-mm diameter) of the outline followed by a crisscross cut, thus producing four skin flaps. The epidermis of the four flaps was then cut away while following the hypodermis superior to the fascia. The area was then trimmed with fine forceps and iris scissors. During surgery, the area was kept moist by applying drops of PBS containing 1% penicillin/streptomycin. The bottom portion of the chamber was put in place, and the top was carefully positioned on the cut side so that the window and the circular incision were fitted. Antibiotic ointment was applied at this time. The three screws that hold the chamber together were then positioned into the chamber holes and tightened so that the skin was not pinched, to avoid diminished circulation. Animals were placed under a heating lamp for several days. Tumor angiogenesis within the window was monitored by microscopy. Tumor blood vessels developed in the window within 1 week.

Five mice were studied in each of the treatment groups (radiation only, PI3Kδ selective inhibitor only, and PI3Kδ selective inhibitor plus radiation). When indicated, a PI3Kδ selective inhibitor in accordance with the invention (25 mg/kg) was injected i.p. about 30 minutes before irradiation. Tissues under the vascular windows were treated with 2 Gy of X-rays using 80 kVp (Pantak X-ray Generator). The window frames were marked with coordinates, which were used to photograph the same microscopic field each day. Vascular windows were photographed using a 4× objective to obtain a 40× total magnification. Color photographs were used to catalogue the appearance of blood vessels on days 0-7. Photographs were scanned into Adobe® Photoshop® software, and vascular center lines were positioned by ImagePro® software and verified by an observer blinded to the treatment groups. Tumor blood vessels were quantified by the use of ImagePro software, which quantifies the vascular length density of blood vessels within the microscopic field. Center lines were verified before summation of the vascular length density. The mean and 95% confidence intervals of vascular length density for each treatment group were calculated, and variance was analyzed by the General Linear Models and Bonferroni t test.

Five mice were treated in each of the treatment groups (radiation only, PI3Kδ selective inhibitor only, and PI3Kδ selective inhibitor plus radiation), and the VLD was quantified at various times after treatment. At 48 hours after treatment with a combination of PI3Kδ selective inhibitor and 2 Gy radiation, VLD in tumors was significantly reduced to about 8% of that at 0 hours (p<0.01). In comparison, tumors treated with either 2 Gy or PI3Kδ selective inhibitor alone showed lesser but still measurable reductions in VLD, to about 75% and to about 84% of the value at the 0 hour time point, respectively. VLD in untreated mice showed no significant change in 48 hours. These data demonstrate a greater than additive effect of the combination of a PI3Kδ selective inhibitor and radiation as determined by multiplying the fractional vascular density achieved by each modality treatment individually to yield an expected value if the effects of each treatment modality were additive.

This example demonstrates that administration of a PI3Kδ selective inhibitor destroys the vasculature supplying LLC tumors with blood and nutrients in greater amounts than radiation therapy alone. This example further demonstrates that administration of a PI3Kδ selective inhibitor potentiates radiation-induced destruction of tumor vasculature, as compared to either therapy alone (p=0.011).

### Example 8

### Tumor Growth Delay is Enhanced by Administering a PI3Kδ Selective Inhibitor

To determine whether a PI3Kδ selective inhibitor in accordance with the invention affects tumor growth delay, mice bearing hind limb tumors were treated with a PI3Kδ selective inhibitor or vehicle control. Tumor volumes were measured using skin calipers.

C57BL/6 mice received subcutaneous injections in the right thigh with 10⁶ viable cells of a murine glioblastoma (GL261) or lung carcinoma (LLC) suspended in 0.2 mL of a 0.6% solution of agarose. The GL261 cell line was obtained from Dr. Yancy Gillespie (University of Alabama, Birmingham, Ala.). GL261 cells were maintained in DMEM with Nutrient Mixture F-12 1:1 (Life Technologies, Inc.) with 7% FCS, 0.5% penicillin-streptomycin, and 1% sodium pyruvate. Lewis Lung Carcinoma (LLC) cells were obtained as previously described, and were maintained in DMEM supplemented with 10% FCS and 1% penicillin-streptomycin. All cells were incubated at 37° C. in a 5% CO₂ incubator.

The mice were stratified into four groups on day 1 (vehicle, PI3Kδ selective inhibitor alone, vehicle+18 Gy radiation, and PI3Kδ selective inhibitor+18 Gy radiation). An equal number of large- and intermediate-sized tumors were present in each group. Mouse tumors were stratified into groups so that the mean tumor volume of each group was comparable. The mean tumor volumes were 240 mm³ (range 205-262) on day 1 for LLC and 260 mm³ (range 240-285) for GL261. These volumes were reached at 12 and 14 days following implantation for LLC and GL261, respectively.

When indicated, the mice received i.p. injections of about 25 mg/kg of PI3Kδ selective inhibitor or drug vehicle approximately 30 minutes prior to each 3 Gy dose of radiation, for a total of six administrations.

A total dose of 18 Gy radiation was administered to the appropriate mice in six fractionated doses of 3 Gy on days 1-6. Both the inhibitor and radiation were discontinued after day 6.

Irradiated mice were immobilized in Lucite chambers, and the entire mouse body was shielded with lead except for the tumor-bearing hind limb. Tumor volumes were measured three times weekly using skin calipers as described previously [Geng et al., supra; Schueneman et al., supra]. The volumes were calculated from a formula (a×b×c/2) that was derived from the formula for an ellipsoid (πd³/6). Data were calculated as the percentage of original (day 1) tumor volume and graphed as fractional tumor volume±SEM for each treatment group.

The mean fold-increases in tumor volumes in five mice in each of the treatment groups were determined. The number of days for GL261 tumor growth to increase by 5-fold as compared to day 1 tumor size was 8, 12, 19 and 33 days for each treatment group, respectively.

Both LLC and GL261 tumors showed a significant increase in tumor growth delay when a PI3Kδ selective inhibitor was added prior to daily administration of 3 Gy as compared with administration of either agent alone (p<0.05).

This example demonstrates that administration of a PI3Kδ selective inhibitor enhances tumor growth delay when compared to a control. This example further demonstrates that administration of a PI3Kδ selective inhibitor potentiates radiation-induced tumor growth delay, as compared to either therapy alone. These data demonstrate a greater than additive effect of the combination of a PI3Kδ selective inhibitor and radiation on tumor growth delay.

### Example 9

### Tumor Blood Flow is Reduced by Administering a PI3Kδ Selective Inhibitor

To determine whether tumor growth delay correlated with a reduction in tumor blood flow, power Doppler ultrasonography was used to monitor tumor blood flow.

Blood flow within the LLC and GL261 tumors was quantified by Power Doppler imaging after the administration of the third fraction of irradiation described in Example 8. Tumor blood flow was imaged with a 10-5 MHz linear Entos probe attached to an HDI 5000 (probe and HDI 5000 from ATL/Philips, Bothell, Wash.) as previously described [Geng et al., supra; Schueneman et al., supra]. Images were obtained with the power gain set to 82%. A 20-frame cineloop sweep (a cineloop is a rapid recording of multiple ultrasound frames encompassing several cardiac cycles, i.e., a digital video of the pulsating vessel) of the entire tumor was obtained with the probe perpendicular to the long axis of the lower extremity along the entire length of the tumor. Intensity of blood flow was imaged as areas of color and quantified using HDI-lab software (ATL/Philips). This software allows direct evaluation of the generated cineloop. The color area was recorded for the entire tumor. Five mice were entered into each treatment group (control, radiation alone, PI3Kδ selective inhibitor alone, and PI3Kδ selective inhibitor and radiation). Values for color area were averaged for each tumor set, and treated groups were compared with controls with the unpaired Student t test.

Reduced blood flow in tumors treated with a PI3Kδ selective inhibitor and radiation correlated with the improved tumor growth delay described in Example 8. The decrease in tumor blood flow for each of the treatment groups (control, radiation alone, PI3Kδ selective inhibitor alone, and PI3Kδ selective inhibitor and radiation) was determined. Blood flow within GL261 tumors was reduced to approximately 40% for the radiation alone treatment group, approximately 25% for the PI3Kδ selective inhibitor alone treatment group, and approximately 15% for the PI3Kδ selective inhibitor and radiation treatment group, with respect to the control group.

This example demonstrates that administration of a PI3Kδ selective inhibitor inhibits a tumor blood supply when compared to a control. This example further demonstrates that administration of a PI3Kδ selective inhibitor in combination with radiation reduces a tumor blood supply by a greater amount than radiation alone (p<0.05).

### Example 10

### Statistical Analysis

The General Linear Model (logistic-regression analysis) was used to test for associations between the numbers of apoptotic cells present in culture, clonogenic survival, tumor blood flow, and tumor volumes. The Bonferroni method was used to adjust the overall significant level equals to 5% for the multiple comparisons in this study. All statistical tests were two-sided, and differences were considered statistically significant for p<0.05. SAS software version 8.1 (SAS Institute, Inc., Cary, N.C.) was used for all statistical analyses.

What is claimed is:

1. A method for inhibiting angiogenesis, comprising:
selectively inhibiting phosphoinositide 3-kinase delta (PI3Kδ) activity in endothelial cells to inhibit angiogenesis.

2. The method of claim 1, wherein inhibiting comprises administering an amount of a PI3Kδ selective inhibitor effective to inhibit angiogenesis.

3. A method for inhibiting endothelial cell migration, comprising:
selectively inhibiting phosphoinositide 3-kinase delta (PI3Kδ) activity in endothelial cells to inhibit endothelial cell migration.

4. The method of claim 3, wherein inhibiting comprises administering an amount of a PI3Kδ selective inhibitor effective to inhibit endothelial cell migration.

5. A method for inhibiting tumor growth, comprising:
selectively inhibiting phosphoinositide 3-kinase delta (PI3Kδ) activity in endothelial cells to inhibit tumor growth.

6. The method of claim 5, wherein inhibiting comprises administering an amount of a PI3Kδ selective inhibitor effective to inhibit tumor growth.

7. A method for reducing tumor vasculature formation or repair, comprising:
selectively inhibiting phosphoinositide 3-kinase delta (PI3Kδ) activity in endothelial cells to reduce tumor vasculature formation or repair.

8. The method of claim 7, wherein inhibiting comprises administering an amount of a PI3Kδ selective inhibitor effective to reduce tumor vasculature formation or repair.

9. A method for inhibiting endothelial tubule formation, comprising:
selectively inhibiting phosphoinositide 3-kinase delta (PI3Kδ) activity in endothelial cells to inhibit endothelial tubule formation.

10. The method of claim 9, wherein inhibiting comprises administering an amount of a PI3Kδ selective inhibitor effective to inhibit endothelial tubule formation.

11. A method for reducing tumor mass, comprising:
selectively inhibiting phosphoinositide 3-kinase delta (PI3Kδ) activity in endothelial cells to reduce tumor mass.

12. The method of claim 11, wherein inhibiting comprises administering an amount of a PI3Kδ selective inhibitor effective to reduce tumor mass.

13. The method of claim 1, wherein said inhibiting is in vitro.

14. The method of claim 1, wherein said inhibiting is in vivo.

15. A method for treating or preventing an indication involving angiogenesis, comprising:
selectively inhibiting phosphoinositide 3-kinase delta (PI3Kδ) activity in endothelial cells to inhibit angiogenesis in an individual in need thereof.

16. The method of claim 15, wherein inhibiting comprises administering an amount of a PI3Kδ selective inhibitor effective to inhibit angiogenesis in an individual in need thereof.

17. The method of claim 15, wherein the indication involving angiogenesis is cancer.

18. The method of claim 17, wherein the cancer is selected from the group consisting of solid tumors, hematological cancers, and lymphomas.

19. The method of claim 1, further comprising administering a cytotoxic therapeutic.

20. The method of claim 19, wherein the cytotoxic therapeutic is administered in an amount effective to increase Akt phosphorylation.

21. The method of claim 19, wherein the cytotoxic therapeutic comprises radiation.

22. The method of claim 19, wherein the cytotoxic therapeutic comprises an anti-angiogenic compound.

23. The method of claim 19, wherein the cytotoxic therapeutic is selected from the group consisting of photodynamic therapy and radiofrequency ablation.

24. The method of claim 19, wherein the cytotoxic therapeutic comprises a chemotherapeutic.

25. The method of claim 24, wherein the chemotherapeutic is a DNA-damaging agent selected from the group consisting of alkylating agents and intercalating agents.

26. The method of claim 15, wherein the indication involving angiogenesis is selected from the group consisting of retinopathy, age-related macular degeneration, arthritis, psoriasis, atherosclerosis, and endometriosis.

27. A method for enhancing apoptosis in endothelial cells, comprising:
selectively inhibiting phosphoinositide 3-kinase delta (PI3Kδ) activity in endothelial cells to enhance apoptosis in endothelial cells.

28. The method of claim 27, wherein inhibiting comprises administering an amount of a PI3Kδ selective inhibitor effective to enhance apoptosis in endothelial cells.

29. The method of claim 28, further comprising administering the PI3Kδ selective inhibitor in combination with radiation to enhance apoptosis in endothelial cells.

30. The method of claim 28, further comprising administering the PI3Kδ selective inhibitor in combination with a chemotherapeutic agent to enhance apoptosis in endothelial cells.

31. The method of claim 28, further comprising administering the PI3Kδ selective inhibitor in combination with an anti-angiogenic agent to enhance apoptosis in endothelial cells.

32. A method for increasing the therapeutic index of radiation therapy, comprising:
administering a combination comprising radiation and an amount of a phosphoinositide 3-kinase delta (PI3Kδ) selective inhibitor effective to increase the therapeutic index of the radiation.

33. A method for increasing the therapeutic index of a chemotherapeutic agent, comprising:
administering a combination comprising a chemotherapeutic agent and an amount of a phosphoinositide 3-kinase delta (PI3Kδ) selective inhibitor effective to increase the therapeutic index of the chemotherapeutic agent.

34. A method for increasing the therapeutic index of an anti-angiogenic agent, comprising:
administering a combination comprising an anti-angiogenic agent and an amount of a phosphoinositide 3-kinase delta (PI3Kδ) selective inhibitor effective to increase the therapeutic index of the anti-angiogenic agent.

35. The method of claim 27, further comprising administering a cytoxic therapy selected from the group consisting of photodynamic therapy and radiofrequency ablation.

36. The method of claim 28, wherein the PI3Kδ selective inhibitor is administered to an individual in need thereof.

37. The method of claim 36, wherein the individual has cancer.

38. The method of claim 37, wherein the cancer is selected from the group consisting of solid tumors, hematological cancers, and lymphomas.

39. A method for reducing highly vascularized tissues, comprising:
selectively inhibiting phosphoinositide 3-kinase delta (PI3Kδ) activity in endothelial cells to reduce vascular growth or vascular repair of a highly vascularized tissue.

40. The method of claim 39, wherein inhibiting comprises administering to an individual an amount of a PI3Kδ selective inhibitor effective to reduce vascular growth or vascular repair of a highly vascularized tissue.

41. The method of claim 40, wherein the highly vascularized tissue is adipose tissue.

42. The method of claim 40, wherein the highly vascularized tissue is retinal tissue.

43. The method of claim 2, wherein the PI3Kδ selective inhibitor is administered in an amount effective to inhibit Akt phosphorylation.

44. The method of claim 2, wherein the PI3Kδ selective inhibitor is a compound having formula (I) or pharmaceutically acceptable salts and solvates thereof: 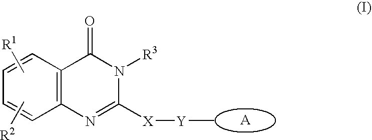
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

45. The method of claim 2, wherein the PI3Kδ selective inhibitor is selected from the group consisting of:
2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydro-quinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N{2-[5-Methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}-5-methyl-2-(9H-purin-6-ylsulfanylmethyl)-3H-quin-azolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3, 5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimidin-3-yl)-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 5-methyl-3-phenyl-2-[1-(9H-purin-6-ylamino)-propyl]-3H-quinazolin-4-one; 2-[1-(2-fluoro-9H-purin-6-ylamino)-ethyl]-5-methyl-3-phenyl-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-difluoro-phenyl)-5-methyl-3H-quinazolin-4-one; 3-(2,6-difluoro-phenyl)-2-[1-(2-fluoro-9H-purin-6-ylamino)-ethyl]-5-methyl-3H-quinazolin-4-one; 3-(2,6-difluoro-phenyl)-5-methyl-2-[1-(7H-pyrrolo[2,3-d]pyrimidin-4-ylamino)-ethyl]-3H-quinazolin-4-one; 2-[1-(2-amino-9H-purin-6-ylamino)-propyl]-5-methyl-3-phenyl-3H-quinazolin-4-one; 5-methyl-3-phenyl-2-[1-(7H-pyrrolo[2,3-d]pyrimidin-4-ylamino)-propyl]-3H-quinazolin-4-one; 2-[1-(2-fluoro-9h-purin-6-ylamino)-propyl]-5-methyl-3-phenyl-3h-quinazolin-4-one; 5-methyl-3-phenyl-2-[1-(9H-purin-6-ylamino)-ethyl]-3H-quinazolin-4-one; 2-[1-(2-amino-9H-purin-6-ylamino)-ethyl]-5-methyl-3-phenyl-3H-quinazolin-4-one; 2-[2-benzyloxy-1-(9H-purin-6-ylamino)-ethyl]-5-methyl-3-phenyl-3H-quinazolin-4-one; 2-[1-(2-amino-9H-purin-6-ylamino)-2-benzyloxy-ethyl]-5-methyl-3-phenyl-3H-quinazolin-4-one; 2-[2-benzyloxy-1-(7H-pyrrolo[2,3-d]pyrimidin-4-ylamino)-ethyl]-5-methyl-3-phenyl-3H-quinazolin-4-one; 2-[2-benzyloxy-1-(2-fluoro-9H-purin-6-ylamino)-ethyl]-5-methyl-3-phenyl-3H-quinazolin-4-one; 3-(4-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(4-fluoro-phenyl)-5-methyl-3H-quinazolin-4-one; 3-(4-fluoro-phenyl)-2-[1-(2-fluoro-9H-purin-6-ylamino)-ethyl]-5-methyl-3H-quinazolin-4-one; 3-(4-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(7H-pyrrolo[2,3-dipyrimidin-4-ylamino)-ethyl]-3H-quinazolin-4-one; 3-(3-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-fluoro-phenyl)-5-methyl-3H-quinazolin-4-one; 3-(3-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(9H-purin-6-yl)-pyrrolidin-2-yl]-3H-quinazolin-4-one; 2-[2-hydroxy-1-(9H-purin-6-ylamino)-ethyl]-5-methyl-3-phenyl-3H-quinazolin-4-one; 5-methyl-3-phenyl-2-[phenyl-(9H-purin-6-ylamino)-methyl]-3H-quinazolin-4-one; 2-[(2-amino-9H-purin-6-ylamino)-phenyl-methyl]-5-methyl-3-phenyl-3H-quinazolin-4-one; 2-[(2-fluoro-9H-purin-6-ylamino)-phenyl-methyl]-5-methyl-3-phenyl-3H-quinazolin-4-one; 5-methyl-3-phenyl-2-[phenyl-(7H-pyrrolo[2,3-d]pyrimidin-4-ylamino)-methyl]-3H-quinazolin-4-one; 5-fluoro-3-phenyl-2-[1-(9H-purin-6-ylamino)-ethyl]-3H-quinazolin-4-one; 2-[1-(2-amino-9H-purin-6-ylamino)-ethyl]-5-fluoro-3-phenyl-3H-quinazolin-4-one; 2-[1-(2-amino-9H-purin-6-ylamino)-ethyl]-5-chloro-3-phenyl-3H-quinazolin-4-one; [5-(5-methyl-4-oxo-3-phenyl-3,4-dihydro-quinazolin-2-yl)-5-(9H-purin-6-ylamino)-pentyl]-carbamic acid benzyl ester; [5-(2-amino-9H-purin-6-ylamino)-5-(5-methyl-4-oxo-3-phenyl-3,4-dihydro-quinazolin-2-yl)-pentyl]-carbamic acid benzyl ester; [4-(5-methyl-4-oxo-3-phenyl-3,4-dihydro-quinazolin-2-yl)-4-(9H-purin-6-ylamino)-butyl]-carbamic acid benzyl ester; [4-(2-amino-9H-purin-6-ylamino)-4-(5-methyl-4-oxo-3-phenyl-3,4-dihydro-quinazolin-2-yl)-butyl]-carbamic acid benzyl ester; 3-phenyl-2-[1-(9H-purin-6-ylamino)-ethyl]-3H-quinazolin-4-one; 2-[5-amino-1-(9H-purin-6-ylamino)-pentyl]-5-methyl-3-phenyl-3H-quinazolin-4-one); 2-[5-amino-1-(2-amino-9H-purin-6-ylamino)-pentyl]-5-methyl-3-phenyl-3H-quinazolin-4-one; 2-[1-(2-amino-9H-purin-6-ylamino)-ethyl]-3-(2,6-Dimethyl-phenyl)-5-methyl-3H-quinazolin-4-one; 3-(2,6-dimethyl-phenyl)-5-methyl-2-[1-(9H-purin-6-ylamino)-ethyl]-3H-quinazolin-4-one; 5-morpholin-4-ylmethyl-3-phenyl-2-[1-(9H-purin-6-ylamino)-ethyl]-3H-quinazolin-4-one; 2-[1-(2-amino-9H-purin-6-ylamino)-ethyl]-5-morpholin-4ymethyl-3-phenyl-3H-quinazolin-4-one; 2-[4-amino-1-(2-amino-9H-purin-6-ylamino)-butyl]-5-methyl-3-phenyl-3H-quinazolin-4-one; 6-fluoro-3-phenyl-2-[1-(9H-purin-6-ylamino)-ethyl]-3H-quinazolin-4-one; 2-[1-(2-amino-9H-purin-6-ylamino)-ethyl]-6-fluoro-3-phenyl-3H-quinazolin-4-one; 2-[2-tert-butoxy-1-(9H-purin-6-ylamino)-ethyl]-5-methyl-3-phenyl-3H-quinazolin-4-one; 3-(3-methyl-phenyl)-5-methyl-2-[1-(9H-purin-6-ylamino)-ethyl]-3H-quinazolin-4-one; 2-[1-(2-amino-9H-purin-6-ylamino)-ethyl]-3-(3-methyl-phenyl)-5-methyl-3H-quinazolin-4-one; 3-(3-chloro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-chloro-phenyl)-5-methyl-3H-quinazolin-4-one; 2-[1-(2-amino-9H-purin-6-ylamino)-2-hydroxy-ethyl]-5-methyl-3-phenyl-3H-quinazolin-4-one; 2-[1-(2-amino-9H-purin-6-ylamino)-ethyl]-3-(3-fluoro-phenyl)-3H-quinazolin-4-one; 2-[1-(2-amino-9H-purin-6-ylamino)-ethyl]-3-(2,6-difluoro-phenyl)-3H-quinazolin-4-one; 2-[1-(2-amino-9H-purin-6-ylamino)-propyl]-5-fluoro-3-phenyl-3H-quinazolin-4-one; 5-chloro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 3-phenyl-2-[1-(9H-purin-6-ylamino)-ethyl]-5-trifluoromethyl-3H-quinazolin-4-one; 3-(2,6-difluoro-phenyl)-5-methyl-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-propyl]-3-(2,6-difluoro-phenyl)-5-methyl-3H-quinazolin-4-one; 2-[1-(2-amino-9H-purin-6-ylamino)-ethyl]-3-(2,6-difluoro-phenyl)-5-methyl-3H-quinazolin-4-one; 3-(3,5-dichloro-phenyl)-5-methyl-2-[1-(9H-purin-6-ylamino)-ethyl]-3H-quinazolin-4-one; 3-(2,6-dichloro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-dichloro-phenyl)-5-methyl-3H-quinazolin-4-one; 5-chloro-3-phenyl-2-[1-(9H-purin-6-ylamino)-propyl]-3H-quinazolin-4-one; 2-[1-(2-amino-9H-purin-6-ylamino)-propyl]-5-chloro-3-phenyl-3H-quinazolin-4-one; 5-methyl-3-phenyl-2-[1-(9H-purin-6-ylamino)-butyl]-3H-quinazolin-4-one; 2-[1-(2-amino-9H-purin-6-ylamino)-butyl]-5-methyl-3-phenyl-3H-quinazolin-4-one; 2-[1-(2-amino-9H-purin-6-ylamino)-ethyl]-3-(3,5-dichloro-phenyl)-5-methyl-3H-quinazolin-4-one; 5-methyl-3-(3-morpholin-4-ylmethyl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-ylmethyl-phenyl)-3H-quinazolin-4-one; 2-[1-(5-bromo-7H-pyrrolo[2,3-d]pyrimidin-4-ylamino)-ethyl]-5-methyl-3-phenyl-3H-quinazolin-4-one; 5-methyl-2-[1-(5-methyl-7H-pyrrolo[2,3-d]pyrimidin-4-ylamino)-ethyl]-3-phenyl-3H-quinazolin-4-one; 2-[1-(5-fluoro-7H-pyrrolo[2,3-d]pyrimidin-4-ylamino)-ethyl]-5-methyl-3-phenyl-3H-quinazolin-4-one; 2-[2-hydroxy-1-(9H-purin-6-ylamino)-ethyl]-3-phenyl-3H-quinazolin-4-one; 3-(3,5-difluoro-phenyl)-5-methyl-2-[1-(9H-purin-6-ylamino)-propyl]-3H-quinazolin-4-one; 2-[1-(2-amino-9H-purin-6-ylamino)-propyl]-3-(3,5-difluoro-phenyl)-5-methyl-3H-quinazolin-4-one; 3-(3,5-difluoro-phenyl)-2-[1-(9H-purin-6-ylamino)-ethyl]-3H-quinazolin-4-one; 2-[1-(5-bromo-7H-pyrrolo[2,3-d]pyrimidin-4-ylamino)-ethyl]-3-(3-fluoro-phenyl)-5-methyl-3H-quinazolin-4-one; 3-(3-fluoro-phenyl)-5-methyl-2-[1-(5-methyl-7H-pyrrolo[2,3-d]pyrimidin-4-ylamino)-ethyl]-3H-quinazolin-4-one; 3-phenyl-2-[1-(9H-purin-6-ylamino)-propyl]-3H-quinazolin-4-one; 2-[1-(2-amino-9H-purin-6-ylamino)-ethyl]-3-(3,5-difluoro-phenyl)-3H-quinazolin-4-one; 2-[1-(2-amino-9H-purin-6-ylamino)-propyl]-3-phenyl-3H-quinazolin-4-one; 6,7-difluoro-3-phenyl-2-[1-(9H-purin-6-ylamino)-ethyl]-3H-quinazolin-4-one; 6-fluoro-3-(3-fluoro-phenyl)-2-[1-(9H-purin-6-ylamino)-ethyl]-3H-quinazolin-4-one; 2-[4-diethylamino-1-(9H-purin-6-ylamino)-butyl]-5-methyl-3-phenyl-3H-quinazolin-4-one; 5-fluoro-3-phenyl-2-[1-(9H-purin-6-ylamino)-propyl]-3H-quinazolin-4-one; 3-phenyl-2-[1-(9H-purin-6-ylamino)-ethyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-2-[1-(2-fluoro-9H-purin-6-ylamino)-ethyl]-3-phenyl-3H-quinazolin-4-one; 3-(3-fluoro-phenyl)-2-[1-(9H-purin-6-ylamino)-ethyl]-3H-quinazolin-4-one; 5-chloro-3-(3,5-difluoro-phenyl)-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 3-(2,6-difluoro-phenyl)-2-[1-(9H-purin-6-ylamino)-ethyl]-3H-quinazolin-4-one; 5-Methyl-3-phenyl-2-[3,3,3-trifluoro-1-(9H-purin-6-ylamino)-propyl]-3H-quinazolin-4-one; 3-(3-hydroxy-phenyl)-5-methyl-2-[1-(9H-purin-6-ylamino)-ethyl]-3H-quinazolin-4-one; 3-(3-methoxy-phenyl)-5-methyl-2-{1-[9H-purin-6-ylamino]-ethyl}-3H-quinazolin-4-one; 3-[3-(2-dimethylamino-ethoxy)-phenyl]-5-methyl-2-{1-[9H-purin-6-ylamino]-ethyl}-3H-quinazolin-4-one; 3-(3-cyclopropylmethoxy-phenyl)-5-methyl-2-{1-[9H-purin-6-ylamino]-ethyl}-3H-quinazolin-4-one; 5-methyl-3-(3-prop-2-ynyloxy-phenyl)-2-{1-[9H-purin-6-ylamino]-ethyl}-3H-quinazolin-4-one; 2-{1-[2-amino-9H-purin-6-ylamino]ethyl}-3-(3-hydroxyphenyl)-5-methyl-3H-quinazolin-4-one; 2-{1-[2-amino-9H-purin-6-ylamino]ethyl}-3-(3-methoxyphenyl)-5-methyl-3H-quinazolin-4-one; 2-{1-[2-amino-9H-purin-6-ylamino]ethyl}-3-(3-cyclopropylmethoxy-phenyl)-5-methyl-3H-quinazolin-4-one; 2-{1-[2-amino-9H-purin-6-ylamino]ethyl}-5-methyl-3-(3-prop-2-ynyloxy-phenyl)-3H-quinazolin-4-one; 3-(3-ethynyl-phenyl)-5-methyl-2-[1-(9H-purin-6-ylamino)-ethyl]-3H-quinazolin-4-one; 3-{5-methyl-4-oxo-2-[1-(9H-purin-6-ylamino)-ethyl]-4H-quinazolin-3-yl}-benzonitrile; 3-{5-methyl-4-oxo-2-{1-[9H-purin-6-ylamino)-ethyl]4H-quinazolin-3-yl}-benzamide; 3-(3-acetyl-phenyl)-5-methyl-2-{1-[9H-purin-6-ylamino]-ethyl}3H-quinazolin-4-one; 2-(3-(5-methyl-4-oxo-2-{1-[9H-purin-6-ylamino]-ethyl}4H-quinazolin-3-yl-phenoxy acetamide; 5-methyl-2-{1-[9H-purin-6-ylamino]-ethyl}-3-[3-(tetrahydropuran-4-yloxy)-phenyl]-3H-quinazolin-4-one; 3-[3-(2-methoxy-ethoxy)-phenyl]-5-methyl-2-[1-(9H-purin-6-ylamino)-ethyl]-3H-quinazolin-4-one; 6-fluoro-2-[1-(9H-purin-6-ylamino)ethyl]-3-[3-(tetrahydro-pyran-4-yloxy)-phenyl]-3H-quinazolin-4-one; 3-[3-(3-dimethylamino-propoxy)-phenyl]-5-methyl-2-[1-(9H-purin-6-ylamino)-ethyl]-3H-quinazolin-4-one; 2-[1-(2-amino-9H-purin-6-ylamino)-ethyl]-3-(3-ethynyl-phenyl)-5-methyl-3H-quinazolin-4-one; 3-{2-[1-(2-amino-9H-purin-6-ylamino)-ethyl]-5-methyl-4-oxo-4H-quinazolin-3-yl}-benzonitrile; 3-{2-[1-(2-amino-9H-purin-6-ylamino)-ethyl]-5-methyl-4-oxo-4H-quinazolin-3-yl}-benzamide; 3-{2-[1-(2-amino-9H-purin-6-ylamino)-ethyl]-5-methyl-4-oxo4H-quinazolin-3-yl}-benzamide; 5-methyl-3-(3-morpholin-4-yl-phenyl)-2-[1-(9H-purin-6-ylamino)-ethyl]-3H-quinazolin-4-one; 2-[l-(2-amino-9H-purin-6-ylamino)-ethyl]-5-methyl-3-(3-morpholin-4-yl-phenyl)-3H-quinazolin-4-one; 2-[1-(2-amino-9H-purin-6-ylamino)-ethyl]-3-[3-(2-methoxy-ethoxy)-phenyl]-5-methyl-3H-quinazolin-4-one; 2-[1-(2-amino-9H-purin-6-ylamino)-ethyl]-3-[3-(2-dimethylamino-ethoxy)-phenyl]-5-methyl-3H-quinazolin-4-one; 2-[1-(2-amino-9H-purin-6-ylamino)-but-3-ynyl]-5-methyl-3-phenyl-3H-quinazolin-4-one; 2-[1-(2-amino-9H-purin-6-ylamino)-but-3-ynyl]-5-methyl-3-phenyl-3H-quinazolin-4-one; 5-chloro-3-(3,5-difluoro-phenyl)-2-[1-(9H-purin-6-ylamino)-ethyl]-3H-quinazolin-4-one; 2-[l-(2-amino-9H-purin-6-ylamino)-propyl]-5-chloro-3-(3,5-difluoro-phenyl)-3H-quinazolin-4-one; 2-[1-(2-amino-9H-purin-6-ylamino)-ethyl]-5-chloro-3-(3,5-difluoro-phenyl)-3H-quinazolin-4-one; 3-(3,5-difluoro-phenyl)-6-fluoro-2-[1-(9H-purin-6-ylamino)-ethyl]-3H-quinazolin-4-one; 5-chloro-3-(2,6-difluoro-phenyl)-2-[1-(9H-purin-6-ylamino)-propyl]-3H-quinazolin-4-one; 2-[1-(2-amino-9H-purin-6-ylamino)-propyl]-5-chloro-3-(2,6-difluoro-phenyl)-3H-quinazolin-4-one; 5-methyl-3-phenyl-2-[1-(9H-purin-6-yloxy)-ethyl]-3H-quinazolin-4-one; and, pharmaceutically acceptable salts and solvates thereof.

46. An article of manufacture comprising a phosphoinositide 3-kinase delta (PI3Kδ) selective inhibitor and a label indicating a method according to any one of claim 1.

47. Use of a composition comprising at least one phosphoinositide 3-kinase delta (PI3Kδ) selective inhibitor in the manufacture of a medicament for treating or preventing an indication involving angiogenesis.
